(19) United States
(12) Patent Application Publication
(10) Pub. No.: US 2025/0302588 A1
(43) Pub. Date: Oct. 2, 2025

(54) PRESCRIPTION BASED ORTHODONTIC TREATMENT PLATFORM

(71) Applicant: LightForce Orthodontics, Inc., Burlington, MA (US)

(72) Inventors: Alexander Yarmarkovich, Swampsott, MA (US); Amos Benninga, Lexington, MA (US); Jinho Lee, Belmont, MA (US); Matthew Wong, Somerville, MA (US)

(73) Assignee: LightForce Orthodontics, Inc., Burlington, MA (US)

(21) Appl. No.: 19/094,588

(22) Filed: Mar. 28, 2025

Related U.S. Application Data
(60) Provisional application No. 63/571,958, filed on Mar. 29, 2024.
(60) Provisional application No. 63/744,611, filed on Jan. 13, 2025.

(51) Int. Cl. A61C 7/00; G06T 7/00

(52) U.S. Cl. CPC A61C 7/002; G06T 7/00; A61C 2007/004; G06T 2207/30036

(57) ABSTRACT

Orthodontic treatment planning involves determining the desired treatment positions for a patient's teeth. The desired treatment positions may include both aesthetic and clinical considerations that describe the spatial relationship of teeth relative to each other. The inventors have recognized and appreciated that existing techniques, for orthodontic treatment planning, struggle to provide suitable orthodontic treatment plans without substantial involvement from orthodontic clinicians.

### CROSS-REFERENCE TO RELATED APPLICATIONS

This application claims the benefit of priority under 35 U.S.C. § 119 (e) to U.S. Provisional Patent Application Ser. No. 63/571,958, filed on Mar. 29, 2024, and titled “PRESCRIPTION BASED ORTHODONTIC TREATMENT PLATFORM,” and U.S. Provisional Patent Application Ser. No. 63/744,611, filed on Jan. 13, 2025, and titled “PRESCRIPTION BASED ORTHODONTIC TREATMENT PLATFORM,” each of which is incorporated by reference herein in its entirety.

### BACKGROUND

Orthodontic procedures involve orthodontic appliances such as braces, which apply static mechanical forces to the teeth to induce bone remodeling and facilitate alignment. Orthodontic treatment planning may utilize 3D models of a patient's teeth to create a treatment plan for the patient, which may, for instance, include determining where to place brackets for a set of braces.

### SUMMARY

Some embodiments relate to a method for use by an orthodontic treatment platform for generating an orthodontic treatment plan, the method comprising: executing the orthodontic treatment platform using at least one computer hardware processor to perform: obtaining an initial treatment configuration comprising anatomical representations of a patient's teeth; obtaining an initial prescription, the initial prescription comprising spatial coordinates for modeling the position of a patient's teeth, the spatial coordinates being relative to a canonical coordinate system; determining an alignment of the patient's teeth based on the initial treatment configuration and the initial prescription; outputting the alignment of the patient's teeth through a graphical user interface; receiving an updated spatial coordinate for one or more of the patient's teeth through the graphical user interface; and generating an updated prescription by updating the one or more spatial coordinates of the initial prescription based on the updated spatial coordinate.

Some embodiments relate to a method for use by an orthodontic treatment platform for updating an orthodontic treatment plan, the method comprising: executing the orthodontic treatment platform using at least one computer hardware processor to perform: receiving an updated target tooth position based on an adjustment to a planned target tooth position, the planned target tooth position being associated with the orthodontic treatment plan; updating one or more spatial coordinates of a prescription based on the received adjustment, the one or more spatial coordinates being relative to a canonical coordinate system; determining an updated orthodontic treatment plan based on the updated one or more spatial coordinates; and displaying the updated orthodontic treatment plan through the graphical user interface.

Some embodiments relate to a method for use by an orthodontic treatment platform for determining an alignment of patient teeth based on an orthodontic prescription using a plurality of alignment operations, the method comprising: executing the orthodontic treatment platform using at least one computer hardware processor to perform: obtaining an initial treatment configuration comprising anatomical representations of a patient's teeth; obtaining an adjustment to a target tooth position associated with the orthodontic treatment plan; obtaining a prescription, the prescription comprising spatial coordinates for modeling the position of a patient's teeth, the spatial coordinates being relative to a canonical coordinate system; and determining a plurality of alignment operations, based on the prescription, to align the patient's teeth to a treatment configuration, the treatment configuration.

Some embodiments relate to a method for using a trained machine learning (ML) model to determine an orthodontic treatment plan configuration, the method comprising: executing the orthodontic treatment platform using at least one computer hardware processor to perform: obtaining a first treatment configuration and prescription; and processing the first treatment configuration and the prescription using the trained ML model to determine a second treatment configuration, the processing comprising: encoding three-dimensional spatial data as respective tooth data; determining an arrangement for the teeth using the trained ML model to process the encoded three-dimensional spatial data; determining canonical coordinates for the teeth based on the determined arrangement; and determining tooth positions based on the first treatment configuration and the canonical coordinates.

### BRIEF DESCRIPTION OF THE DRAWINGS

FIG. 1 illustrates an example of an orthodontic treatment platform environment for generating orthodontic treatment plans, in accordance with some embodiments of the technology described herein.

FIG. 2 illustrates an example of an orthodontic treatment platform for generating orthodontic treatment plans, in accordance with some embodiments of the technology described herein.

FIG. 3 is an example of an orthodontic treatment platform for determining an orthodontic prescription based on orthodontic practitioners positioning of teeth.

FIG. 4A is a flowchart of an illustrative process for use by an orthodontic treatment platform for generating an orthodontic treatment plan, in accordance with some embodiments of the technology described herein.

FIG. 4B is a flowchart of an illustrative process for use by an orthodontic treatment platform for updating an orthodontic treatment plan, in accordance with some embodiments of the technology described herein.

FIG. 4C is a flowchart of an illustrative process for use by an orthodontic treatment platform for determining an alignment of patient teeth based on an orthodontic prescription using a plurality of alignment operations, in accordance with some embodiments of the technology described herein.

FIG. 4D is a flowchart of an illustrative process for use by an orthodontic treatment platform for determining tooth positions based on canonical coordinates, in accordance with some embodiments of the technology described herein.

FIG. 5A illustrates an example of an orthodontic treatment platform for determining an orthodontic treatment plan using an orthodontic prescription, in accordance with some embodiments of the technology described herein.

FIG. 5B illustrates another example of an orthodontic treatment platform 530 for determining an orthodontic treatment plan using an orthodontic prescription, in accordance with some embodiments of the technology described herein.

FIG. 6 illustrates a trained machine learning model architecture used for training for determining tooth alignment in connection with an orthodontic treatment plan, in accordance with some embodiments of the technology described herein.

FIG. 7 illustrates the validation loss and training loss for an example of a trained machine learning model, in accordance with some embodiments of the technology described herein.

FIG. 8A illustrates a first example of initial, final, and predicted positions for teeth in a front and top view of a patient's mouth, in accordance with some embodiments of the technology described herein.

FIG. 8B illustrates a second example of initial, final, and predicted positions for teeth in a front and top view of a patient's mouth, in accordance with some embodiments of the technology described herein.

FIG. 8C illustrates a third example of initial, final, and predicted positions for teeth in a front and top view of a patient's mouth, in accordance with some embodiments of the technology described herein.

FIG. 8D illustrates a fourth example of initial, final, and predicted positions for teeth in a front and top view of a patient's mouth, in accordance with some embodiments of the technology described herein.

FIG. 9 illustrates an orthodontic arrangement with canonical horizontal and canonical vertical reference planes, in accordance with some embodiments of the technology described herein.

FIG. 10 illustrates a treatment configuration displayed as part of a graphical user interface (GUI) for user review and/or adjustment, in accordance with some embodiments of the technology described herein.

FIG. 11 illustrates an example of input controls that may be used in connection with a GUI for adjusting the position and/or orientation of a tooth, in accordance with some embodiments of the technology described herein.

FIG. 12 illustrates an example of an initial treatment configuration based on a neutral prescription, in accordance with some embodiments of the technology described herein.

FIG. 13 illustrates an example of a tooth orientation with a tip applied to the tooth, in accordance with some embodiments of the technology described herein.

FIG. 14 illustrates an example of a GUI for midline and horizontal plane detection, in accordance with some embodiments of the technology described herein.

FIG. 15 illustrates an example of a GUI for tooth axes configuration, in accordance with some embodiments of the technology described herein.

FIG. 16 illustrates an example of a GUI for arch setup, in accordance with some embodiments of the technology described herein.

FIG. 17 illustrates an example of a GUI for showing an initial prescription, in accordance with some embodiments of the technology described herein.

FIG. 18A illustrates an example of a GUI for showing the initial rotation for a patient's teeth, in accordance with some embodiments of the technology described herein.

FIG. 18B illustrates an example of a GUI for rotation a tooth, in accordance with some embodiments of the technology described herein.

FIG. 19 illustrates an example of a GUI for adjusting the in/out distance of a tooth, in accordance with some embodiments of the technology described herein.

FIG. 20 illustrates a second example of a GUI for adjusting the in/out distance of a patient's teeth, in accordance with some embodiments of the technology described herein.

FIG. 21 illustrates a second example of a GUI for adjusting the IPR/spacing distance of a patient's teeth, in accordance with some embodiments of the technology described herein.

FIG. 22 illustrates an example of a GUI for adjusting the height of the teeth relative to an archform, in accordance with some embodiments of the technology described herein

FIG. 23 illustrates an example of a GUI for adjusting the tilt of the teeth, in accordance with some embodiments of the technology described herein.

FIG. 24 illustrates an example of a GUI for adjusting the torque of the teeth, in accordance with some embodiments of the technology described herein.

FIG. 25 illustrates an example implementation of a computer system 2500 that may be used in connection with any of the embodiments described herein.

### DETAILED DESCRIPTION

Orthodontic treatment planning involves determining the desired treatment positions for a patient's teeth. The desired treatment positions may include both aesthetic and clinical considerations that describe the spatial relationship of teeth relative to each other. The inventors have recognized and appreciated that existing techniques, for orthodontic treatment planning, struggle to provide suitable orthodontic treatment plans without substantial involvement from orthodontic clinicians.

The inventors have recognized and appreciated that manual methods for determining the arrangement of teeth in an orthodontic treatment plan may be prohibitively time consuming such that it can be a limiting step of treatment planning. During manual planning of the arrangement of teeth, an orthodontic practitioner arranges the teeth in their respective positions and then reviews the arrangement to check whether it meets the aesthetic and/or clinical requirements for the orthodontic treatment. Upon determining that an arrangement does not meet the aesthetic or clinical requirements, an orthodontic practitioner must then rearrange teeth to remedy the deficiency. However, when moving teeth in the orthodontic treatment plan, movements applied to a particular tooth may introduce new spacings between the adjacent teeth and/or change the patient's smile or bite. Accordingly, after moving one tooth, it may become necessary to move additional teeth to meet the aesthetic or clinical requirements. This can, effectively, create a cascading effect on surrounding teeth when moving just one single tooth. Therefore, orthodontic treatment planning may require numerous iterations, resulting in a time-intensive planning process.

The inventors have further recognized and appreciated that the automated methods for determining the arrangement of teeth in an orthodontic treatment plan struggle to provide suitable results which can be implemented without manual modification from an orthodontic practitioner. For example, automated methods for arranging the teeth for use with an orthodontic treatment plan may fail to produce suitable positions for the teeth, which meet the aesthetic and/or clinical criteria. Therefore, manual adjustments may remain a prohibitively time-consuming aspect of treatment. In particular, manual adjustments to one tooth do not result in corresponding movements from the other teeth, such as to maintain a set configuration (e.g., spacing and archshape) between the unadjusted teeth. For example, movements along one direction can trigger changes along other dimensions and parameters, such as the interproximal distance. Each of these changes may make it difficult to maintain a desired archshape for the teeth.

Thus, to improve orthodontic treatment planning, the inventors have developed an orthodontic treatment platform that implements orthodontic prescriptions using canonical reference positions such that orthodontic treatment planning may be executed easily, efficiently and with high precision by an orthodontic treatment platform. Accordingly, the use of orthodontic prescriptions may function as building blocks for orthodontic planning to enable more efficient treatment customization for doctors and other practitioners. In response to providing anatomical data from the patient to the orthodontic treatment platform, a three-dimensional arrangement of the teeth is determined that corresponds to the final positions of the teeth in the orthodontic treatment plan. The final positions may then be provided to an orthodontic technician through a graphical user interface such that the technician may review and edit the final positions. Upon receiving edits to the final tooth positions, the canonical reference positions are updated. Through the use of the canonical reference positions, updates to the final position of one tooth may be implemented while maintaining the spatial relationships between the teeth, such that the patient's other teeth do not need to be individually adjusted (e.g., to avoid unintentional changes to the spatial dimensions and/or parameters of the orthodontic treatment plan). For example, use of the canonical reference positions can prevent gaps from being formed between neighboring teeth, after adjusting the position of a target tooth, by providing spatial relationships between the teeth and the reference positions. Through the use of spatial relationships, the positional and orientational relationship are maintained when individual teeth move. Similarly, through the use of the orthodontic prescriptions and orthodontic planning modules, the orthodontic practitioners may be able to specify a desired orthodontic outcome without needing to manually align each tooth to produce the specified outcome.

Accordingly, some embodiments provide for a method for use by an orthodontic treatment platform for generating an orthodontic treatment plan, the method comprising: executing the orthodontic treatment platform using at least one computer hardware processor to perform: obtaining an initial treatment configuration comprising anatomical representations of a patient's teeth; obtaining an initial prescription, the initial prescription comprising spatial coordinates for modeling the position of a patient's teeth, the spatial coordinates being relative to a canonical coordinate system; determining an alignment of the patient's teeth based on the initial treatment configuration and the initial prescription; outputting the alignment of the patient's teeth through a graphical user interface; receiving an updated spatial coordinate for one or more of the patient's teeth through the graphical user interface; and generating an updated prescription by updating the one or more spatial coordinates of the initial prescription based on the updated spatial coordinate.

Some embodiments provide for a method for use by an orthodontic treatment platform for updating an orthodontic treatment plan, the method comprising: executing the orthodontic treatment platform using at least one computer hardware processor to perform: receiving an updated target tooth position based on an adjustment to a planned target tooth position, the planned target tooth position being associated with the orthodontic treatment plan; updating one or more spatial coordinates of a prescription based on the received adjustment, the one or more spatial coordinates being relative to a canonical coordinate system; determining an updated orthodontic treatment plan based on the updated one or more spatial coordinates; and displaying the updated orthodontic treatment plan through the graphical user interface.

Some embodiments provide for a method for use by an orthodontic treatment platform for determining an alignment of patient teeth based on an orthodontic prescription using a plurality of alignment operations, the method comprising: executing the orthodontic treatment platform using at least one computer hardware processor to perform: obtaining an initial treatment configuration comprising an anatomical representation of a patient's teeth; obtaining an adjustment to a target tooth position associated with the orthodontic treatment plan; obtaining a prescription, the prescription comprising spatial coordinates for modeling the position of a patient's teeth, the spatial coordinates being relative to a canonical coordinate system; and determining a plurality of alignment operations, based on the prescription, to align the patient's teeth to a treatment configuration, the treatment configuration.

Some embodiments provide for a method for using a trained machine learning model to determine an orthodontic treatment plan configuration, the method comprising: executing the orthodontic treatment platform using at least one computer hardware processor to perform: obtaining a first treatment configuration and prescription; and processing the first treatment configuration and the prescription using the trained ML model to determine a second treatment configuration, the processing comprising: encoding three-dimensional spatial data as respective tooth data; determining an arrangement for the teeth using the trained ML model to process the encoded three-dimensional spatial data; determining canonical coordinates for the teeth based on the plurality of geometries; and determining tooth positions based on the first treatment configuration and the canonical coordinates.

Orthodontic treatment systems may be used to determine orthodontic prescriptions for use in developing an orthodontic treatment plan. As described above, implementing an orthodontic practitioners specified arrangement of the patient's teeth for use in an orthodontic treatment plan may involve time consuming manual input to configure the modules to determine an orthodontic prescription.

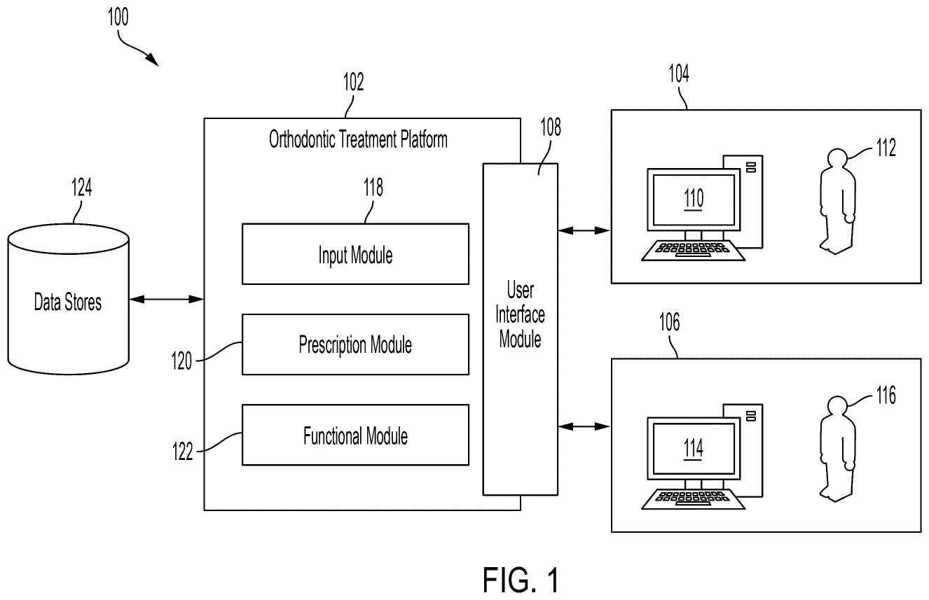

FIG. 1 illustrates an example of an orthodontic treatment platform environment for generating orthodontic treatment plans, in accordance with some embodiments of the technology described herein. Orthodontic treatment platform 100 includes modules to aid in the generation modification and analysis of orthodontic treatment plans for use by orthodontic practitioners. The orthodontic treatment platform facilitates orthodontic treatment planning between orthodontic practitioners.

The orthodontic treatment platform 102 includes input module 118, prescription module 120, functional module 122, and user interface module 108. The prescription module 120 facilitates the generation, modification, and implementation of orthodontic prescriptions. In some embodiments, an orthodontic prescription describes the intended positions for a patient's teeth at the end of treatment. Accordingly, the orthodontic prescription represents the target alignment and positioning of a patient's teeth following the orthodontic treatment procedures included with the orthodontic treatment plan. The orthodontic prescription may have any suitable form for describing the spatial coordinates of the patient's teeth.

The orthodontic treatment platform 102 may send and/or receive information from a data store 124. Orthodontic prescriptions and/or treatment plans may be stored in data store 124. When modifying an orthodontic prescription and/or treatment plan, the orthodontic treatment platform may access the data from the data store for use by the other modules. The orthodontic treatment platform 102 may interface with users of the platform through user interface module 108. As shown in FIG. 1, the orthodontic treatment platform may provide multiple users with access. In some embodiments, users may be orthodontists. For example, a first user 112 may be a first orthodontist working at a first location 104. The first orthodontist 112 may access the user interface module 108 through a user device 110. A second user 116 may be a second orthodontist working at a second location 106. The second orthodontist may access the user interface module 108 through a second user device 114.

In some embodiments, users may be orthodontic technicians. For example, the first user 112 may be a first technician and the second user 116 may be a second technician. In some embodiments, the first technician and the second technician may work in the same office. Accordingly, 104 and 106 may be different treatment rooms in the same office. In some embodiments, the first technician and the second technician may work in separate offices and the first location 104 may be different from the second location 106. In some embodiments, users may be anyone trained in orthodontic design and trained in use of the orthodontic treatment platform. In some embodiments, any combination of users may use the platform. For example, the first user 112 may be an orthodontist and the second user 116 may be an orthodontic technician or may be a specialist in orthodontics design who may aid the orthodontist in designing treatment plans through the orthodontic treatment platform.

In some embodiments, the orthodontic treatment platform 102 may be running on the user device. In such instances, the orthodontic treatment platform 102 communicate with a server over the internet for retrieving data or execution specific modules of the platform. However, in some embodiments, the orthodontic treatment platform may be running entirely locally on the user device.

The inventors have recognized and appreciated that an orthodontic prescription that represents the spatial coordinates of the teeth relative to canonical reference coordinates without needing to describe the full geometry of the individual teeth within the prescription would improve the efficiency of determining orthodontic treatment plans. Additionally, the implementation of treatment planning modules, which use the orthodontic prescription to identify and track changes to the spatial coordinates of the teeth, provides for more accurate and computationally a more efficient determination of orthodontic treatment plans.

In some embodiments, the orthodontic prescription is a matrix having a row for each tooth associated with the orthodontic prescription and a column for each spatial coordinate. The spatial coordinates include three positional coordinates describing the coordinate position of the tooth in the mouth. The spatial coordinates also include three orientational coordinates describing the tip, torque, and rotation of the teeth relative to canonical reference coordinates. For example, the orthodontic prescription may be a matrix having 28 rows and six columns such that spatial coordinates for each of a patient's teeth is represented by the orthodontic prescription. In some embodiments, the orthodontic prescription may include multiple layers to track the changes to the orthodontic prescription or to reflect the different inputs provided by different orthodontic practitioners reviewing a patient's orthodontic treatment plan.

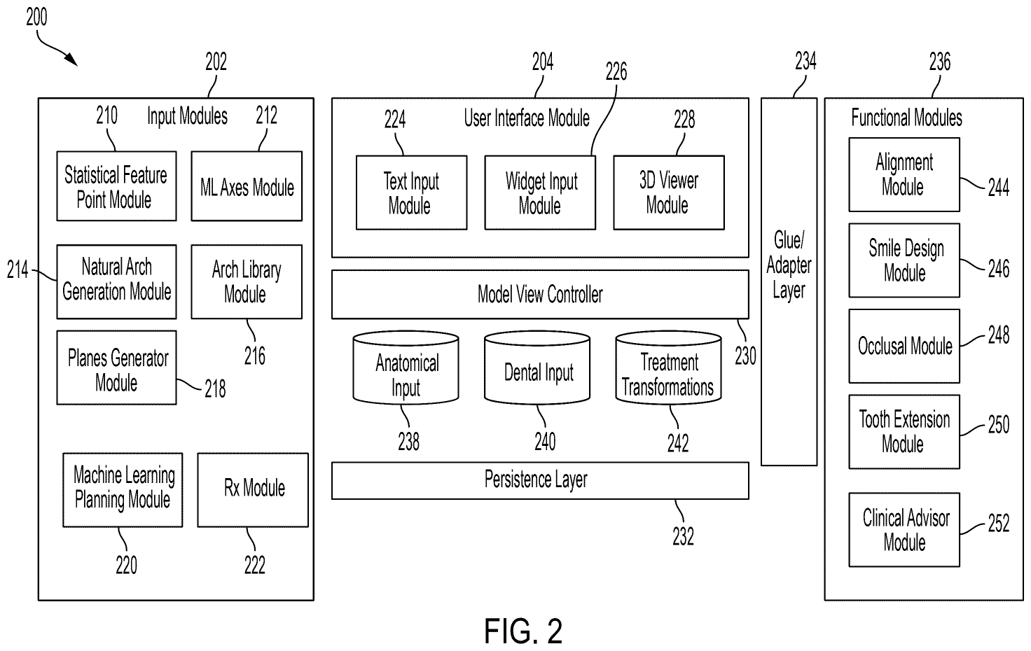

FIG. 2 illustrates an example of an orthodontic treatment platform 200 for generating orthodontic treatment plans, in accordance with some embodiments of the technology described herein. The orthodontic treatment platform includes input modules for generating an initial treatment configuration corresponding to the position of the patient's teeth at the beginning of treatment and or at the beginning of a particular treatment stage. The input modules also include modules to aid in the design of a target treatment configuration representing the positions of a patient's teeth at the intended conclusion of orthodontic treatment.

In some embodiments, the input modules include 202 statistical feature point module 210, machine learning axes module 212, natural arch generation module 214, arch library module 216, plane generator module 218, machine learning planning module 220, and prescription module 222.

The orthodontic treatment platform further includes a user interface module 204 for interfacing with orthodontic practitioners to receive inputs associated with orthodontic treatment planning. In some embodiments, user interface modules include text input module 224, widget input module 226, and 3D viewer module 228.

The orthodontic treatment platform further includes datastores for storing data to be used for the generation and implementation of the orthodontic treatment. The datastores may include an anatomical input datastore 238, dental input datastore 240, and treatment transformation datastore 242.

The orthodontic treatment platform further includes functional modules 236 for implementing modifications and/or refinements to the orthodontic treatment plan. The functional modules may aid in the alignment of teeth for the target treatment configuration as well as providing analysis on the suitability of the alignment for aesthetic and clinical objectives. To the extent that the analysis identifies issues with the alignment, the modules may generate changes to the orthodontic treatment plan to improve the alignment. To identify and/or implement the changes, functional modules 236 include alignment module 244, smile design module 246, occlusion module 248, tooth extension module 250, and clinical advisor module 252.

The orthodontic treatment platform includes a model view controller 230, persistence layer 232, and glue/adapter layer 234 for providing functional connectivity between the modules. The model view controller may manage interfacing with users by generating, updating, and managing the user interface modules. The glue/adapter layer provides a wrapper for organizing and initializing modules to execute processes by the orthodontic treatment platform. The persistence layer provides connectivity between modules and datastores to facilitate the flow of data for processes by the orthodontic treatment platform.

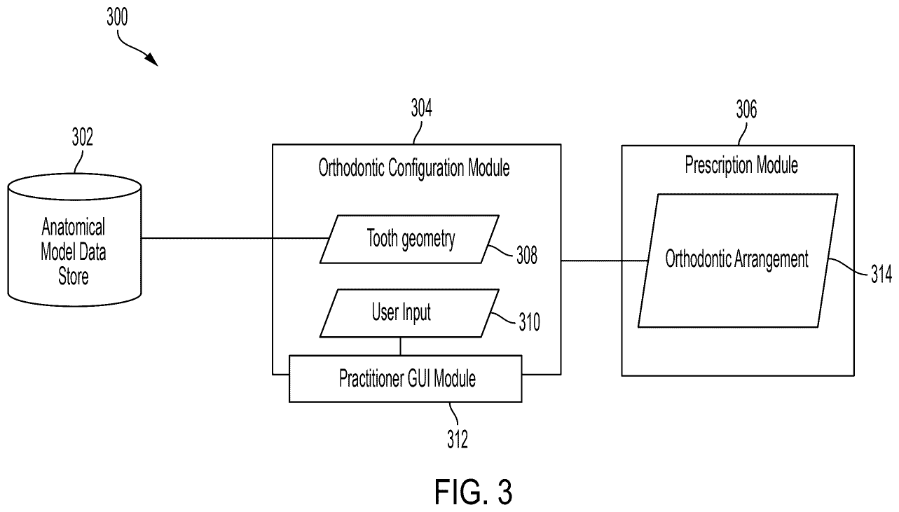

FIG. 3 is an example of an orthodontic treatment platform for determining an orthodontic prescription based on orthodontic practitioners positioning of teeth. An orthodontic configuration module 304 receives a representation of the patient's tooth geometry 308 from an anatomical model datastore 302, wherein the representation may specify a tooth geometry. The tooth geometry includes the shape of the respective teeth to be included in the orthodontic treatment plan as well as the spatial relationship (e.g., the position of the patient's teeth relative to one another) representative of the patient's tooth positions at the start of treatment. The orthodontic configuration module determines an orthodontic treatment configuration, e.g., desired positions for the patient's teeth after treatment has commenced. The desired positions for the orthodontic treatment configuration are received by the orthodontic configuration module from an orthodontic practitioner specifying a desired position for each tooth included in the orthodontic treatment plan. The orthodontic configuration module may interface with a practitioner GUI 312 for receiving the practitioner input 310 specifying the desired positions for the orthodontic treatment plan.

Accordingly, the orthodontic configuration module 304 determines orthodontic translations to be applied to each of the teeth involved in the treatment such that the patient's teeth move from the positions described by the tooth geometry 308 representative of a first treatment configuration to positions described by the second treatment configuration, the second treatment configuration being based on the orthodontic practitioner's input 310.

The positions of the teeth after all translations have been applied is converted into an orthodontic arrangement 314 by the prescription module 306. The orthodontic arrangement may be a numerical prescription, as described herein, that represents the position and orientation of teeth.

The orthodontic configuration module may output a model of the patient's teeth arranged in the second treatment configuration and the determined orthodontic translations. The model of the patient's teeth arranged in the second treatment configuration may be used to review the viability of the model with regard to aesthetics and clinical requirements. The orthodontic translations maybe used to design orthodontic equipment to be used during treatment and the orthodontic process to be employed during the progression of treatment.

Based on the configuration illustrated in FIG. 3, arranging the second treatment configuration may involve meticulous and iterative input from an orthodontic practitioner due to the impact that modifying the placement of one tooth has on the positional relationship with neighboring teeth. For example, changing the position of one target tooth may change the relative height of the target tooth to the neighboring teeth which may impact the clinical bite parameters as it may change the degree of contact between teeth in the upper and lower jaws. Similarly, changing the rotation and or in/out distance of the tooth from an arch line may impact the aesthetic parameters as it may change the visual congruence between the teeth when smiling. One way in which orthodontic technicians may seek to mitigate the impact of moving a single tooth on the surrounding teeth is to select multiple teeth to move at the same time. However, moving multiple teeth as a single unit may still induce discrepancies in alignment as each tooth may have a different positional relationship with the reference points in the mouth with which they should be aligned. For example, moving all of the teeth on the upper right jaw forward may change the relative position of each tooth to an archshape to which the front surfaces of the teeth should be aligned.

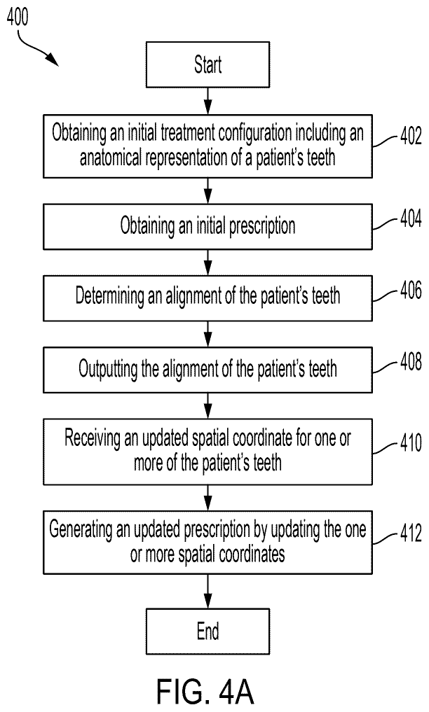

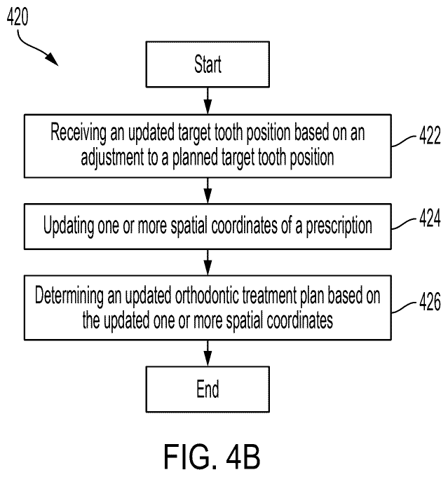

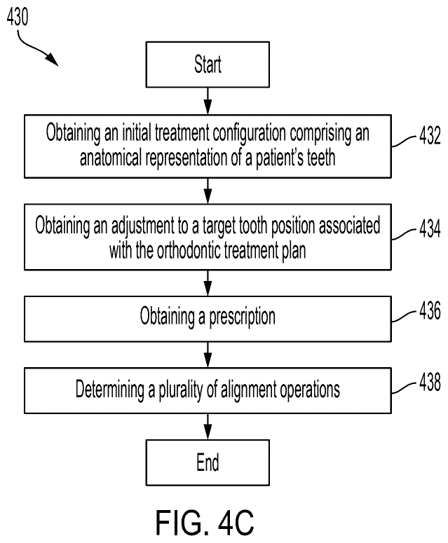

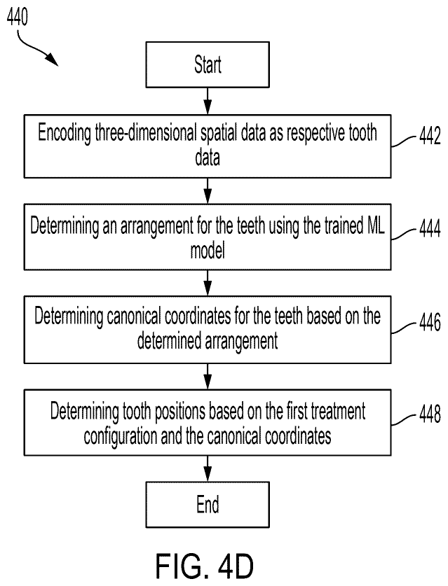

FIG. 4A is a flowchart of an illustrative process 400 for use by an orthodontic treatment platform for generating an orthodontic treatment plan, in accordance with some embodiments of the technology described herein. Process 400 may be executed by any suitable orthodontic treatment platform, such as the orthodontic treatment platform described herein in connection with FIGS. 1-2.

Process 400 starts at act 402 by obtaining an initial treatment configuration including an anatomical representation of a patient's teeth. The anatomical representations include the shapes of the patient's teeth and the positioning of teeth in the patient's mouth. For example, the position of teeth in the patient's mouth may be a relative position for a tooth, such as a position for a tooth that is relative to a neighboring tooth and/or a canonical reference. The canonical reference may be an archform upon which the tooth is to be placed at a relative distance from its neighboring tooth. As another example, the position of teeth in the patient's mouth may be the position of teeth relative to other anatomical features of the patient, such as the patient's facial midline or other anatomical reference features.

In some embodiments, obtaining an initial treatment configuration of a patient's teeth includes acquiring one or more images and/or scans of a patient's teeth. For example, visual images may be acquired of a patient's teeth such that anatomical representations of the patient's teeth may be generated based on the images. Multiple images depicting different perspectives of the patient's teeth may be acquired and used in the generation of the anatomical representation. As another example, scans such as x-rays may be acquired of a patient's teeth such that anatomical representations of the patient's teeth may be generated based on the scans. Multiple scans depicting different perspectives of the patient's teeth may be acquired as used in the generation of the anatomical representation. As another example, both images and scans may be used in combination to generate the anatomical representation of the patient's teeth. Any suitable imaging and/or scanning technique for capturing the shape and/or position may be used, as aspects of the technology described herein are not limited in this respect.

In some embodiments, obtaining an initial treatment configuration includes receiving an orthodontic treatment file that includes anatomical representations of the patient's teeth. The orthodontic treatment file may be any file format that represents the shapes and positions of the patient's teeth. For example, the orthodontic treatment files may be stl files. In some embodiments, multiple orthodontic treatment files may be obtained which represent different perspectives of the patient's teeth.

Process 400 proceeds to act 404 by obtaining an initial prescription, in accordance with some embodiments of the technology described herein. The initial prescription describes the initial positions intended for the teeth in the orthodontic treatment plan. The initial positions intended for the teeth may be a first determination of the tooth positions intended for the patient's teeth at the end of treatment. The initial positions describe the spatial coordinates for modeling the positions of the patient's teeth where the spatial coordinates are measured relative to a canonical coordinate system.

In some embodiments, obtaining the initial prescription includes receiving the initial prescription from a prescription module, such as prescription module 222 described above in connection with FIG. 2. Accordingly, the initial prescription may be retrieved from a non-transitory storage medium. In some embodiments, obtaining the initial prescription includes receiving the initial prescription from a machine learning planning module that uses a trained machine learning module to determine an initial prescription, such as machine learning planning module 220 described above in connection with FIG. 2.

Process 400 proceeds to act 406 by determining an alignment of the patient's teeth, in accordance with some embodiments of the technology described herein. The alignment of the patient's teeth is determined based on the initial treatment configuration and the initial prescription. In some embodiments, the alignment of the patient's teeth may be determined for one or more of six canonical coordinates. The six canonical coordinates may include an in/out distance, spacing, vertical alignment, tip angle, torque angle, and rotation angle—as described herein. The alignment of the patient's teeth may be determined in a prescribed order such that positions are reproducibly determined based on the canonical coordinates. In some embodiments, the rotation is applied first, next the tip is applied, finally the torque is applied. Accordingly, the combination of the common reference coordinates and the prescribed order of orientations provides for the reproducible positioning of the teeth automatically by the process.

Process 400 proceeds to act 408 by outputting the alignment of the patient's teeth, in accordance with some embodiments of the technology described herein. The alignment of the patient's teeth is output through a graphical user interface. The alignment may be represented by graphical depictions of the patient's teeth arranged in a two-dimensional representation of the patient's teeth that represent the positions of the teeth in a three-dimensional environment, in accordance with the alignment. For example, all of the patient's teeth may be shown in the two-dimensional representation of the patient's teeth. As another example, a sub-set of the patient's teeth may be shown in the two-dimensional representation of the patient's teeth. The graphical user interface may further include controls for a user to change the perspective view of the arrangement of the teeth. By changing the perspective view, the graphical user interface may provide a different two-dimensional representation of the arrangement of the patient's teeth from the adjusted perspective selected by the user.

Process 400 proceeds to act 410 by receiving an updated spatial coordinate for one or more of the patient's teeth, in accordance with some embodiments of the technology described herein. The updated spatial coordinate may be provided by a user through the graphical user interface. The graphical user interface includes controls for the user to update the spatial positions of the alignment of the patient's teeth. For example, the positions of the patient's teeth in the alignment can be depicted in a table of positions which may be updated by changing the respective position coordinate in the table. As another example, the positions of the patient's teeth may be updated by selecting arrows corresponding to axis of the teeth and moving the respective tooth along the selected axis.

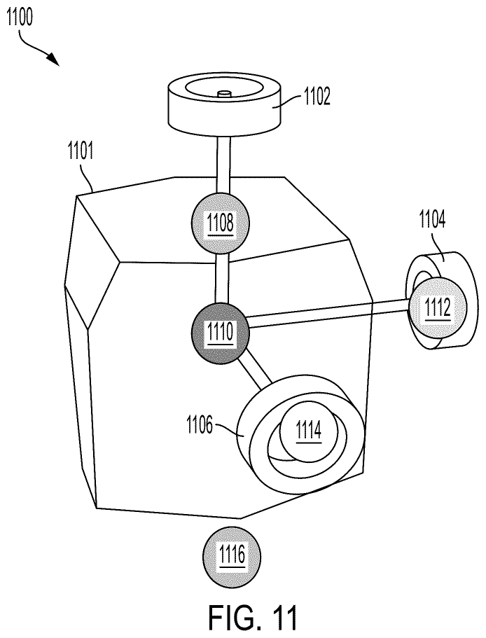

The axes of the teeth are based on the shape of the tooth itself. For example, the position of a tooth axis may be based on feature points of the teeth, as described herein in connection with FIG. 11 below. As another example, the position of a tooth axis may be configured by a user of the orthodontic treatment platform.

Process 400 proceeds to act 412 by generating an updated prescription by updating the one or more spatial coordinates of the initial prescription based on the updated spatial coordinate, in accordance with some embodiments of the technology described herein. In response to receiving an updated spatial coordinate, process 400 updates the prescription. To update the prescription, the updated spatial coordinate is determined relative to the canonical coordinate system. In some embodiments, the updated spatial coordinate received from the graphical user interface may be a differential position, e.g., coordinates corresponding to the difference between the unadjusted spatial coordinate and the updated spatial coordinate. Based on the differential position, the updated prescription is generated by adding the differential position to the spatial coordinates in the alignment of the patient's teeth. In some embodiments, the updated spatial coordinate received from the graphical user interface is a tooth position. The tooth position may be used for the spatial coordinates of the tooth in the prescription. If the coordinates are received in the canonical coordinate system then the canonical coordinates can be directly used in the prescription. If the coordinates are received in a different coordinate system, the coordinates are converted into the canonical coordinate system prior to being used in the prescription.

Following the conclusion of act 412, process 400 concludes. Following the conclusion of process 400, the updated prescription may be used to determine an updated alignment of the patient's teeth and to output the updated alignment for display. In response to receiving additional updates to the spatial coordinates of the patient's teeth, act 412 may be repeated and the updated prescription used as an input to act 406 and 408 to determine an updated alignment and output such as to provide real time updates to the displayed positions of the patient's teeth.

FIG. 4B is a flowchart of an illustrative process 420 for use by an orthodontic treatment platform for updating an orthodontic treatment plan, in accordance with some embodiments of the technology described herein. Prior to the start of process 420 an initial prescription may be used to determine and to display, through a graphical user interface, an alignment of the patient's teeth. Through the graphical user interface, the user may adjust the position of one or more of the patient's teeth, as described above in connection with FIG. 4A. Process 420 may be executed by any suitable orthodontic treatment platform, such as the orthodontic treatment platform described herein in connection with FIGS. 1-2.

Process 420 begins at act 422 by receiving an updated target tooth position based on an adjustment to a planned target tooth position, in accordance with some embodiments of the technology described herein. The planned target tooth position is associated with the orthodontic treatment plan.

Process 420 proceeds to act 424 by updating one or more spatial coordinates of a prescription, in accordance with some embodiments of the technology described herein. The one or more spatial coordinates are updated relative to the canonical coordinate system. The canonical coordinate system includes both position coordinates and orientation coordinates, as described herein. For example, the orientation coordinates may include three coordinates representing a canonical x-position, canonical y-position, and canonical z-position. The orientation coordinates may include three angles: a tip, tilt, and rotation. The orientation angles being measured between the teeth axis and canonical coordinates. In some embodiments, the one or more spatial coordinates may be updated by input provided by a user through the graphical user interface, as described above in connection with FIG. 4A.

In some embodiments, updating the one or more spatial coordinates includes replacing the corresponding position and/or orientation in the prescription with the updated coordinate. In some embodiments, updating the one or more spatial coordinates includes generating a separate prescription. The separate prescription may be stored as an additional layer of an array of coordinates corresponding to the prescription. Accordingly, the record of changes of the prescription may be stored as separate layers of an array providing for a review of particular changes to the prescription. In some embodiments, layers of the prescription are associated with a user of the graphical user interface.

Process 420 proceeds to act 426 by determining an updated orthodontic treatment plan based on the updated one or more spatial coordinates, in accordance with some embodiments of the technology described herein. The updated orthodontic treatment plan is determined for each of the patient's teeth to accommodate the updated target tooth position while maintaining the spatial coordinates specified by the prescription.

Following act 426, process 420 concludes. Following the conclusion of process 420, the updated orthodontic treatment plan is displayed through the graphical user interface. Following display of the updated orthodontic treatment plan, process 420 may start again by receiving another updated target tooth position. By executing process 420 in response to each updated target tooth position received, process 420 may provide real time updates to the displayed positions of the patient's teeth.

FIG. 4C is a flowchart of an illustrative process 430 for use by an orthodontic treatment platform for determining an alignment of patient teeth based on an orthodontic prescription using a plurality of alignment operations, in accordance with some embodiments of the technology described herein. Process 430 may be executed by any suitable orthodontic treatment platform, such as the orthodontic treatment platform described herein in connection with FIGS. 1-2.

Process 430 begins at act 432 by obtaining an initial treatment configuration including an anatomical representation of the patient's teeth, in accordance with some embodiments of the technology described herein. The anatomical representations may include the shapes of the patient's teeth and the positioning of teeth in the patient's mouth, as described above with reference to process 400.

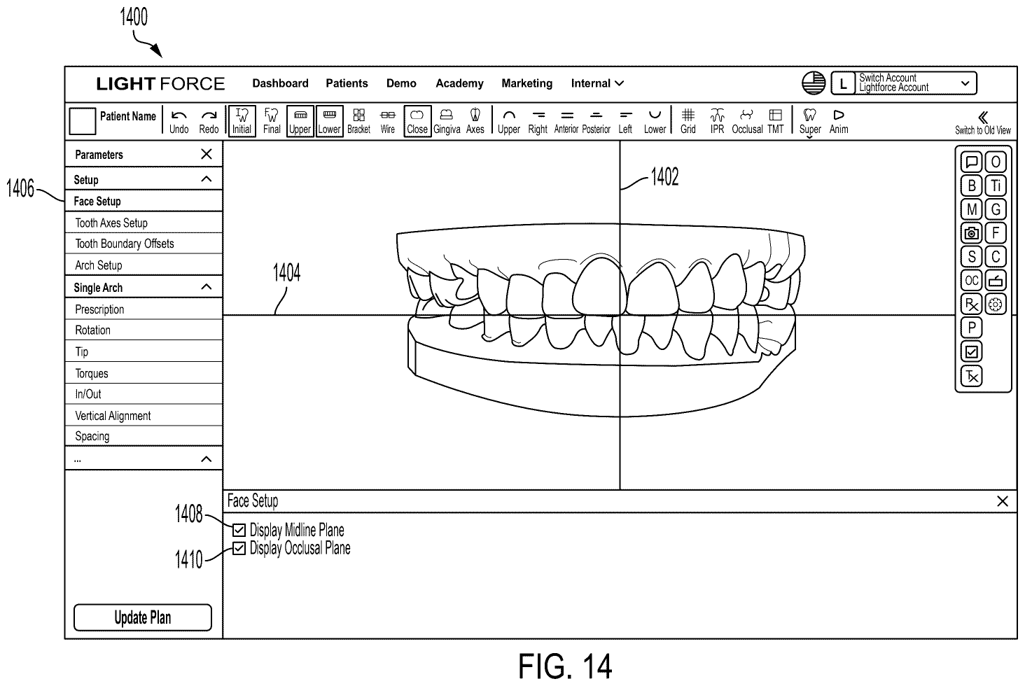

Process 430 proceeds to act 434 by obtaining an adjustment to a target tooth position associated with the orthodontic treatment plan, in accordance with some embodiments of the technology described herein. The adjustment may be obtained in the manner described in connection with process 400 above. In some embodiments, the adjustment may be obtained through a user interface. Examples of user interfaces through which a user may input adjustments to an orthodontic treatment system are shown in FIGS. 14-24.

Process 430 proceeds to act 436 by obtaining a prescription, in accordance with some embodiments of the technology described herein. The prescription includes position coordinates and orientation coordinates, as described herein.

Process 430 proceeds to act 438 by determining a plurality of alignment operations, in accordance with some embodiments of the technology described herein. Determining the alignment operations includes aligning the target tooth to the one or more position coordinates. Determining the alignment operations is executed in a prescribed order such that adjustments are reproducibly applied to facilitate adjustments to the treatment plan. In some embodiments, the plurality of alignment operations is determined for the patient's teeth sequentially from the front teeth to the back teeth.

In some embodiments, determining the alignment operations includes determining the position of the target tooth relative to the other teeth. For example, determining the alignment operations include determining an arch distance between the target tooth position and an archform, determining a spacing between the target tooth position and a neighboring tooth, and determining a vertical offset between the target tooth position and an initial vertical position.

For example, determining a spacing between the target tooth position and a neighboring tooth includes determining a distance between relative treatment features of the tooth. The relative treatment features may be represented by feature points.

Determining the alignment operations may include aligning the height of the teeth to provide for an aesthetic smile. In some embodiments, determining the alignment operations includes determining the vertical positions of the teeth. For example, determining the alignment operations includes aligning the height of each of the teeth in the orthodontic treatment plan such that the vertical offset to the teeth increases from the front teeth to the back teeth. The vertical offset may be measured relative to the horizontal plane.

Following act 438, process 430 concludes.

FIG. 4D is a flowchart of an illustrative process 440 for use by an orthodontic treatment platform for determining tooth positions based on canonical coordinates, in accordance with some embodiments of the technology described herein. Prior to the start of process 440, tooth data for a patient may be received having a number of sampled points representing points on the surfaces of the patient's teeth. In some embodiments, tooth data may be received in a numerical array having a separate set of points for each tooth, where the set of points are each described by an x, y, and z coordinate.

Process 440 starts at act 442 by encoding three-dimensional spatial data as respective tooth data, in accordance with some embodiments of the technology described herein. In some embodiments, process 440 receives tooth data as a set of spatial coordinates associated with particular teeth. The set of spatial coordinates is encoded to generate tooth surfaces. The tooth surfaces are used to represent the geometry of individual teeth.

Process 440 continues to act 444 by determining an arrangement for the teeth using the trained ML model, in accordance with some embodiments of the technology described herein. The trained ML model receives the encoded three-dimensional spatial data as an input and determines an arrangement for the teeth as an output of the trained ML model. In some embodiments, the trained ML model is a trained neural network model.

Process 440 continues to act 446 by determining canonical coordinates for the teeth based on the determined arrangement for the teeth, in accordance with some embodiments of the technology described herein. Canonical coordinates for the teeth are determined based on the arrangement determined by the machine learning model. The canonical coordinates for the teeth may be determined by comparing the tooth positions in the arrangement with canonical x, y, and z axes, as described herein.

Process 440 continues to act 448 by determining tooth positions based on the first treatment configuration and the canonical coordinates, in accordance with some embodiments of the technology described herein. From the treatment configuration and canonical coordinates, a transformation for each tooth is determined to move the tooth from the initial position to the desired position in the treatment configuration. In some embodiments, the transformation is a 4×4 transformation that applies to the initial alignment of individual teeth to get to the desired final alignment.

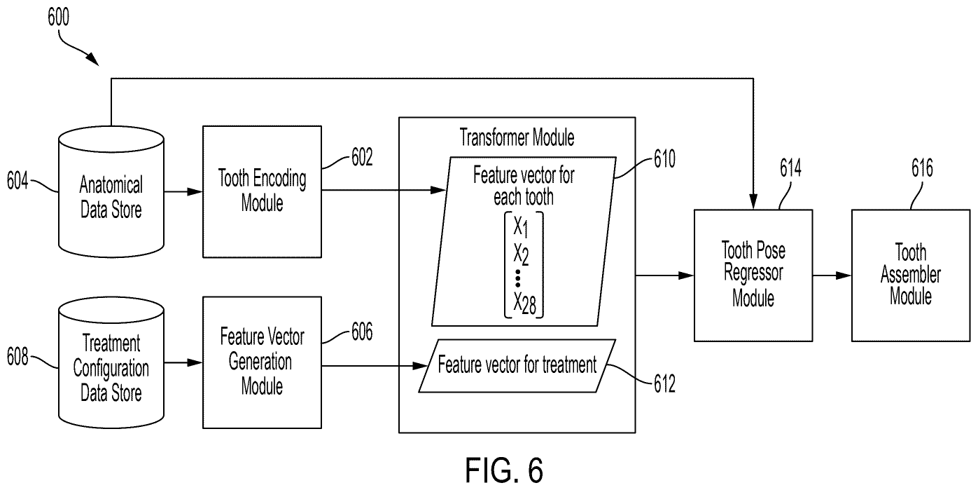

Following act 448, process 440 concludes. One example of a trained machine learning model which may implement a process such as process 440 is described below in connection with FIG. 6. In some embodiments, the trained ML model determines a relative transformation for each tooth which would result in the treatment configuration when applied to the initial alignment.

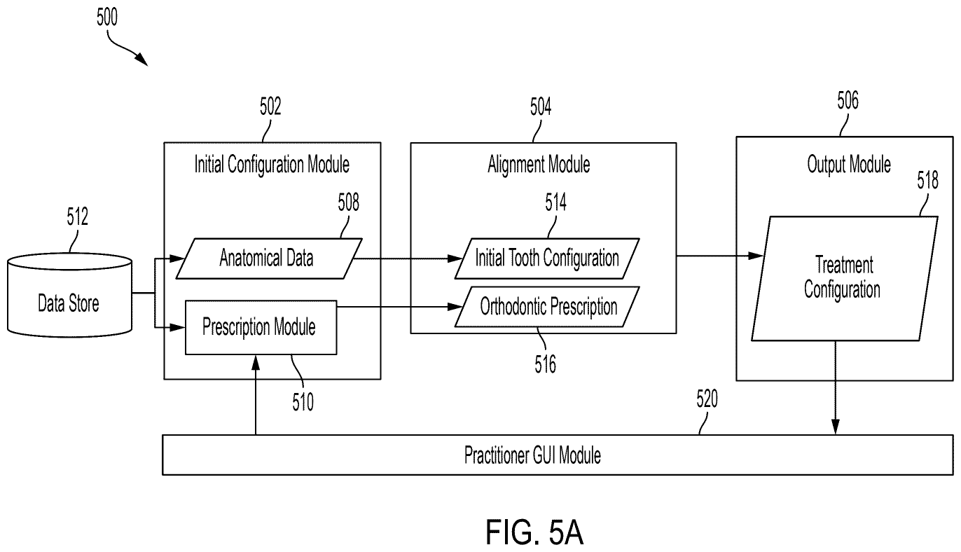

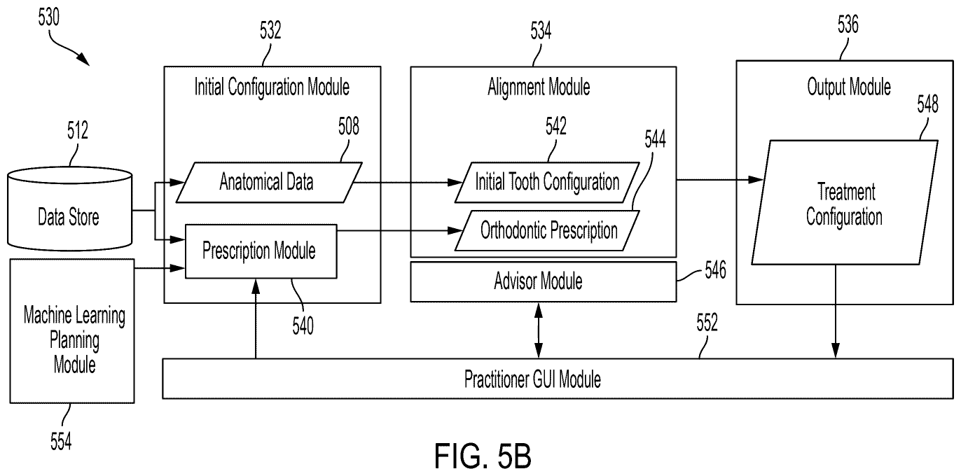

FIG. 5A illustrates an example of an orthodontic treatment platform for determining an orthodontic treatment plan using an orthodontic prescription, in accordance with some embodiments of the technology described herein. The orthodontic treatment platform 500 includes an initial configuration module, an alignment module, an output module, and a practitioner GUI module.

The initial configuration module 502 determines an initial treatment tooth configuration that describes positions for the patient's teeth at the beginning of treatment. The initial tooth configuration may be determined by the initial configuration module using anatomical data 508 of the patient's teeth. For example, anatomical data 508 of the patient's teeth may be received by the initial configuration module in a general three-dimensional file format that describes the geometry of the patient's mouth without associating geometric features with particular teeth. The three-dimensional file format may be received from data store 512, as described below. Accordingly, the initial configuration module may process the anatomical data 508 to generate one or more tooth models corresponding to the shape and position of each tooth to be included in the orthodontic treatment plan.

The one or more tooth models may include geometric features (e.g., specific structures corresponding to the shape of the tooth) extracted from the three-dimensional file format. For example, the one or more tooth models may include particular feature points associated with geometric features relevant to tooth alignment and/or evaluating the quality of orthodontic alignment. In some embodiments, the tooth models may include midline and/or horizontal planes, tooth axes, and feature points.

In some embodiments, the anatomical data may be received from a datastore 512. The datastore may include stored scans of a patient's teeth and mouth. The anatomical data may be stored in any suitable format. In some embodiments, the anatomical data may be stored as several files. For example, as several individual scans of different portions (e.g., different views) of a patient's mouth. As another example, the individual scans may correspond to different imaging and/or scanning modalities, such as visual images and x-rays.

The anatomical data may also be acquired in other ways. In some embodiments, the anatomical data may be received directly from an instrument used to acquire scans of a patient's mouth. In some embodiments, the anatomical data may be uploaded by a user of platform.

The initial configuration module includes a prescription module 510 for receiving an orthodontic prescription for use in determining an orthodontic treatment plan. In some embodiments, the orthodontic prescriptions may be received from the datastore 512, when an existing prescription is used for the orthodontic treatment plan. In some embodiments, the prescription module may receive a prescription through the practitioner GUI module 520. For example, when a new prescription is being provided by an orthodontic practitioner.

The prescription module may receive updates to desired tooth positions for the orthodontic treatment plan and may generate an updated prescription for the orthodontic treatment plan to reflect the changes to the desired tooth positions.

The alignment module 504 determines a treatment configuration for the patient's teeth based on the initial tooth configuration 514 and the orthodontic prescription 516. The alignment module 504 receives the initial tooth configuration 514 and the orthodontic treatment prescription 516 from the initial configuration module. In some embodiments, to determine the treatment configuration, the alignment module 504 aligns the patient's teeth, from the initial tooth configuration, in accordance with the orthodontic prescription.

In some embodiments, the alignment module 504 uses one or more specific alignment processes to determine various aspects of the alignment for the treatment configuration. The specific alignment processes may be implemented as respective modules or submodules of alignment module 504. The one or more specific alignment processes use the orthodontic prescription to place and orient the teeth relative to canonical reference coordinates. Through use of the canonical reference coordinates, the placement of teeth in the treatment configuration for the patient is reproducibly produced from the orthodontic prescription.

The output module 506 receives the treatment configuration from the alignment module and interfaces with practitioner GUI module to provide the treatment configuration to orthodontic practitioners, such that the orthodontic practitioners may review the treatment configuration for aesthetic and clinical requirements. The output module 506 may also analyze the treatment configuration to provide orthodontic practitioners with the parameters for treatment. For example, the output module may check the treatment configuration for indicators related to specific treatment methods such as whether the orthodontic treatment plan includes an interproximal reduction.

Practitioner GUI module 520 provides the treatment configuration 518 to an orthodontic practitioner for review. Practitioner GUI module 520 additionally may receive input from the orthodontic practitioner to modify the position of one or more teeth in the treatment configuration.

In some embodiments, upon receiving input from the orthodontic practitioner, practitioner GUI module 520 provides the practitioner input to the prescription module 510 to update the orthodontic prescription. In response to generating an updated orthodontic prescription-based on the feedback received from the practitioner GUI module—the initial configuration module, alignment module, and output module process the updated orthodontic prescription to modify the treatment configuration 518 displayed to the orthodontic practitioner such that the position of the one or more modified teeth is updated in real time without inducing unintended changes to the patient's other teeth.

FIG. 5B illustrates another example of an orthodontic treatment platform 530 for determining an orthodontic treatment plan using an orthodontic prescription, in accordance with some embodiments of the technology described herein. The orthodontic treatment platform 530 includes initial configuration module 532, alignment module 534, output module 536, practitioner GUI module 552, machine learning planning module 554, and advisor module 546. The initial configuration module alignment module and output module may be configured in the same way as the corresponding modules shown in FIG. 5A, described above.

As shown in FIG. 5B, the orthodontic treatment platform 530 further includes advisor module 546 and machine learning planning module 554 to aid in the review of treatment configurations and the preparation of orthodontic prescriptions, respectively. The advisor module 546 reviews the treatment configuration 548 for aesthetic and/or clinical parameters and upon determining that the treatment configuration deviates from the desired aesthetic and/or clinical parameters, the advisor module may determine one or more modifications to the treatment configuration 548. Upon determining one or more modifications to the treatment configuration, the updates maybe implemented as modifications to the orthodontic prescription 544.

Accordingly, in some embodiments, upon updating the orthodontic prescription—the initial configuration module, alignment module, output module, and practitioner GUI module may implement the one or more modifications to the treatment configuration 548 in real time. The practitioner GUI module 552 may provide the one or more modifications generated by the advisor module 546 to the orthodontic practitioner such that they may choose whether or not to implement the modifications, determined by the advisor module 546.

Trained machine learning planning module 554 determines an orthodontic prescription 544 for use by the prescription module 540 of the initial configuration module 532. The trained machine learning module 554 being configured to receive an anatomical representation of the patient's teeth and, based on training data, determine positions for the patient's teeth to be used as target locations in the treatment configuration. The positions of the patient's teeth and their corresponding orientations may then be used to determine a prescription representative of the configuration produced by the trained machine learning model. The generated orthodontic prescription may then be received by the prescription module 540 to generate a treatment configuration 548 for orthodontic practitioner review through the practitioner GUI module 552.

FIG. 6 illustrates a trained machine learning model architecture used for training for determining tooth alignment in connection with an orthodontic treatment plan, in accordance with some embodiments of the technology described herein. Trained machine learning environment includes tooth encoding module, transformer module, tooth pose regressor module, and tooth assembler module.

The tooth encoding module encodes the tooth geometry from a received format to a feature vector for further processing by subsequent network modules. In some embodiments, the received format is a point cloud and a PointNet architecture is used to encode tooth geometry. In the illustrated example, a 3×3 pose transform TNet, 64×64 feature transform TNet, followed by a shared Multi-Layer Perception (MLP) with layers (64, 128, 1024) is used for encoding. By applying a global max pooling layer, a feature vector size of 1024 for each tooth is obtained (x1 . . . x28 in FIG. 6). In other embodiments, a PointNet++, PointCNN, DGCNN, or PointTransformer may be used in multiple configurations. In some embodiments, the PointNet architecture performed more effective tooth encoding in terms of training speed and prediction accuracy.

In some embodiments, the accuracy of the model is improved by encoding the feature vector, which encodes a desired treatment configuration data provided by a user, and adding it to the encoded tooth features. For example, the original configuration data is processed into a feature vector with length 34 and encoded using an MLP with layers (34, 2116, 1024), the output of which is added to the encoded tooth features prior to being processed by the transformer module.

The transformer module receives the encoded tooth geometry features for the individual teeth and is configured to determine the alignment for the respective teeth. The transformer module architecture is based on a natural language processing architecture. The transformer is configured to treat the role of each tooth's geometry in a tooth alignment problem as similar to that of a word token in a sentence when solving a generic language problem. The transformer module uses the encoded tooth features as input and produces an output having 28 feature vectors corresponding to the input tooth. The 28 output feature vectors (each with the same length as the input (1028)) contain each tooth's encoded geometry as well as the relationship of this tooth with other teeth that are important to the alignment after training. This structure produces effective solutions for the teeth alignment without the use of any jaw-level feature encoding.

In some embodiments, the transformer configuration includes an attention depth of 4 and 4 attention heads. The attention depth and attention heads use each tooth's geometric center for positional encoding with an MLP having layers (3, 128, 1028). Output of the MLP layers is added to the output of each tooth encoder.

Tooth pose regressor module 614 determines the spatial coordinates for each of the teeth in the orthodontic treatment plan. The spatial coordinates being indicative of the pose of each tooth. In some embodiments, a dropout value is used in the layers, except for in the final where a linear activation layer is implemented. For example, the dropout layer may be configured with a dropout value of 0.3.

Tooth assembler module 616 applies the spatial coordinates determined by the tooth pose regressor module 614 to the point cloud points from the initial alignment. The transformed points from the initial alignment are mapped onto the final alignment. A smooth L1 loss between the mapping is used to determine the loss value which is to be minimized during the model training process. In some embodiments, an AdamW optimizer is used to adjust the model parameters to reduce the loss value.

In some embodiments, the trained machine learning model includes between 50 million and 400 million, between 100 million and 400 million, between 100 million and 200 million, or between 125 million and 175 million trainable parameters. For example, the trained machine learning model may include 142 million trainable parameters. In some embodiments, the trained machine learning model may additionally include non-trainable parameters.

To train the machine learning model, a training dataset was used for generating training and validation datasets to evaluate the model after each epoch's training and to optimize the training time and to obtain evaluation metrics.

The output of the trained machine learning model is an estimate of the final alignment based on the initial alignment and the treatment configuration of a new orthodontic treatment plan. In some embodiments, the output of the prediction is 6 DOF spatial parameters for each of 28 teeth. The spatial parameters may be converted into 4×4 transformation matrices and applied to the initial alignment of individual teeth to get the predicted final alignment of all teeth. The inference process of the trained machine learning model is very fast. For example, the trained machine learning model may determine the predicted final arrangement in approximately two seconds on a regular machine without any GPU.

For training the machine learning model, the input geometry of individual teeth and the initial teeth alignment in a treatment case are provided as training data such that, once trained, the machine learning model is configured to estimate the relative transformation of each tooth which would result in the final teeth alignment when applied to the initial alignment. A third dataset, the test dataset, was generated for comparing different models trained with different training datasets. The samples in the three datasets are mutually exclusive and each sample in a dataset contains all three types of processed data items (as described further below) for an actual orthodontics treatment planning case. The composition of the datasets is shown in Table 1, below.

TABLE 1

| Dataset Name | Dataset Composition |
|---|---|
| Training Dataset | 24240 samples (8080 original samples + |
| | 16160 augmented samples) |
| Validation Dataset | 2022 samples (without any augmentation) |
| Test Dataset | 1642 samples (without any augmentation) |

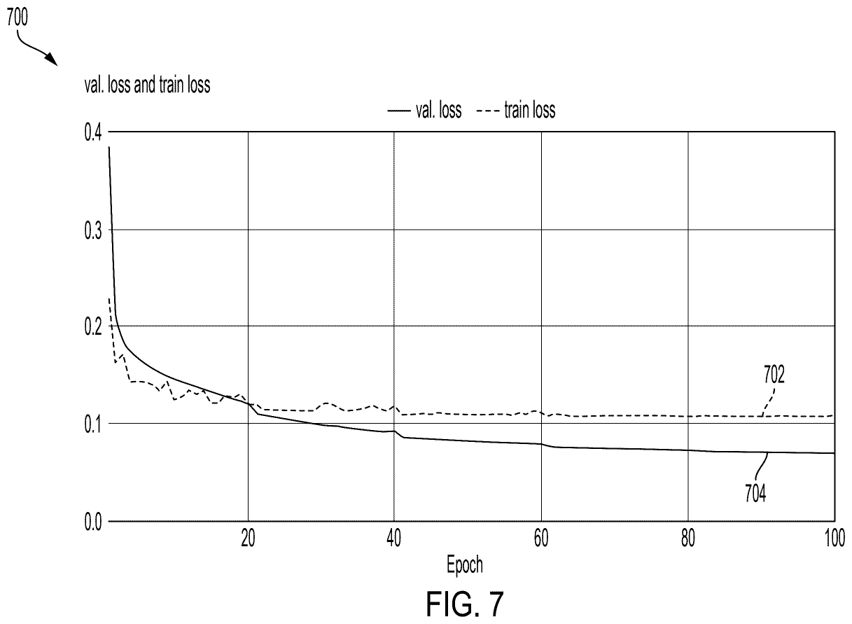

An AdamW optimizer was used to train the model for 100 epochs with initial learning parameter 0.0001 and weight decay 0.01. During training a learning rate scheduler was used which decreased the learning rate by half for every 20 epochs. See FIG. 7 for a chart that shows the training loss and validation loss across the 100 training epochs.

In some embodiments, the samples have a dimension of (28, 400, 3) corresponding to 28 teeth, 400 points per tooth, and 3 spatial coordinates for each point.

In some embodiments, the trained machine learning model is a neural network model configured to learn the relationship between the input geometry and the 6-degree pose parameters (transformation) that would lead each tooth to the final alignment. The supervised learning approach is based on a neural network, which iteratively minimizes the loss function with the repeated feeding of the many input data samples (geometry+treatment configuration) and the known corresponding output data (transformation or pose) for a training dataset. The result of the training process produces an optimized set of weights for the network, which in turn can be used to predict the output pose parameters for the new input data, which has not been a part of the training dataset.

Instead of using direct regression of pose parameters, the final teeth alignment (ground truth) is compared to the output teeth alignment which is reconstructed by the proposed pose parameters during training. The distance of two-point cloud sets is measured and used for the main loss function.

The raw training data includes a 3D geometry of each individual tooth to be included in the orthodontic treatment plan where, for each individual tooth geometry, the position may be described in its own respective coordinate system. For example, the 3D geometry for each individual tooth may be provided as a point cloud. The anatomical data may further include transformations of each tooth to align the 3D geometry to the initial teeth setup. For example, the transformations of each tooth may be used to align the 3D geometry to resemble the original jaw scan of the patient.

The anatomical data may include transformations of each tooth to align the 3D geometry to the final tooth configuration. For example, after receiving manual input from an orthodontic practitioner, the manual inputs may be included with the anatomical data.

The anatomical data may include treatment configuration data. For example, orthodontic practitioner's input on treatment options and/or preferences on aspects of treatment, such as a desired IPR, over jet, etc.

The training data is processed prior to training. The received anatomical data is processed by sampling points from the 3D geometry. In some embodiments, the sampling includes 400 points for each tooth uniformly sampled across the tooth surface. For example, the sampling may use the Farthest Point Sampling technique.

The processing may include determining relative transformations from the initial tooth positions to the final tooth configurations. For example, determining relative transformations may include determining a 3D rotation matrix from the relative transformation to the axis rotation formation such that the rotation can be described using 3 degrees of freedom (e.g., determined from the 3D rotation matrix having 9 degrees of freedom, a representation of the teeth that uses 3 degrees of freedom). In some embodiments, the numerical array used in training has a dimension of (28,6) corresponding to the 28 teeth and the 6 spatial parameters including the 3 axes of rotation and 3 translations.

Additionally, the string values of treatment configuration data may be converted into numerical arrays. For example, the string values representative of the treatment configuration may be expressed as feature vectors. In some embodiments, the numerical array has a length of 34 corresponding to 11 configuration fields.

To augment data for training, two additional training samples were generated for each one original training sample. The data augmentation was performed by random rotation of each tooth in the initial teeth alignment and applying the same amount of rotation to the ground truth of relative transformation.

FIG. 7 illustrates the validation loss and training loss for an example of a trained machine learning model, in accordance with some embodiments of the technology described herein. The accuracy of the trained machine learning model 700 is compared to that of another model generated using a different test dataset, as shown in Table 2.

TABLE 2

| Model | ΔT_{avg} (mm) | Δθ_{avg} (degree) | ADD (mm) |
|---|---|---|---|
| TANet | 0.97 | 6.64 | 1.036 |
| Trained Machine | 0.64 | 5.25 | 0.712 |
| Leaning Model 700 | | | |

In Table 2, shown above, ΔTavg is the mean translation error, Δθavg is the mean rotation error, and ADD is the mean distance error between the predicted alignment and final alignment (each represented as a point cloud).

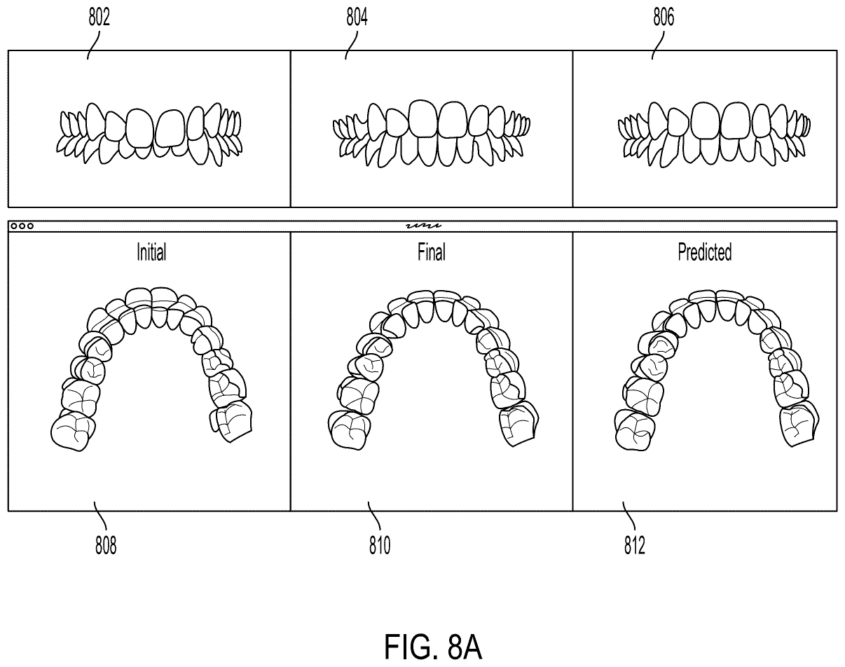

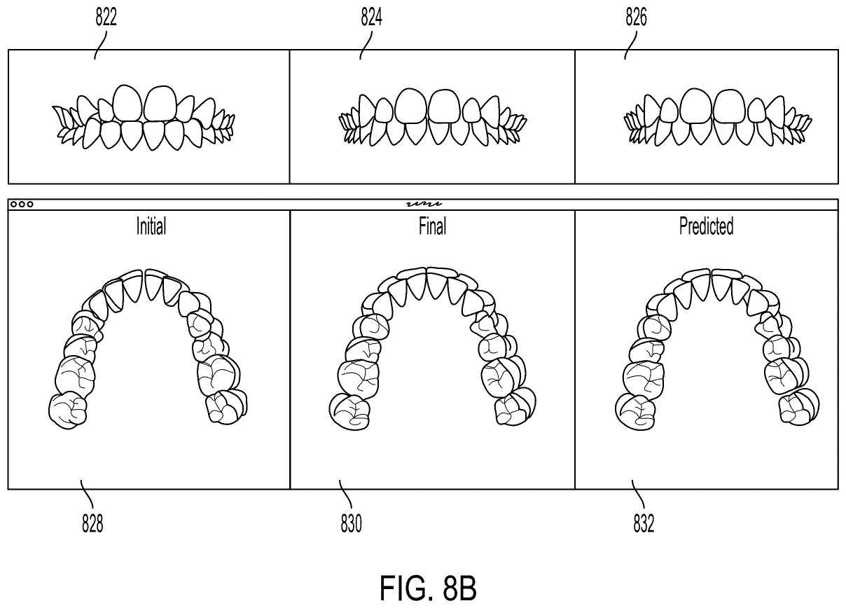

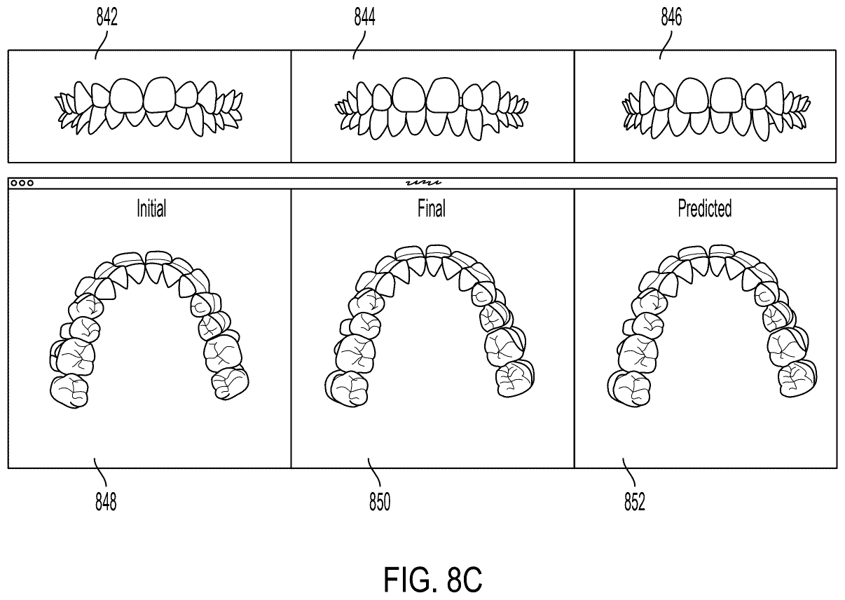

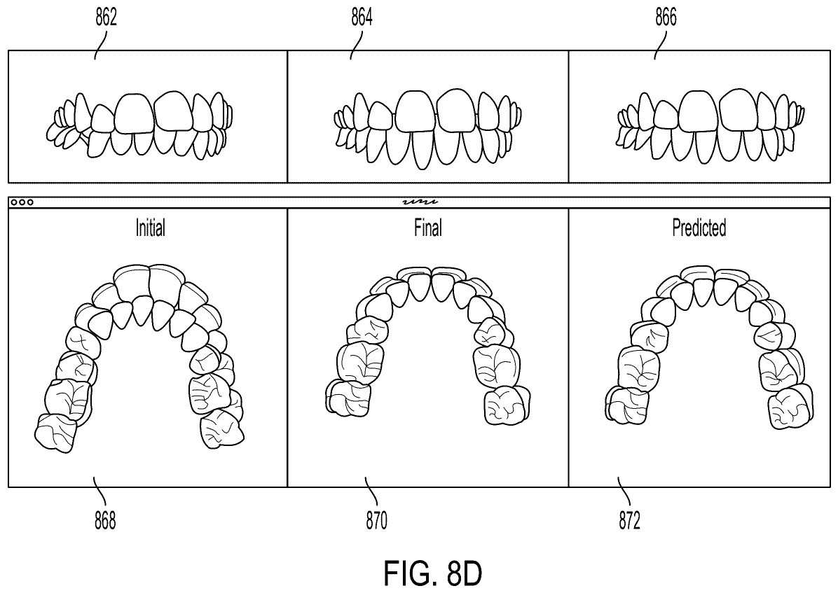

FIGS. 8A-8D are a few examples of the input (initial) and output (predicted), and ground truth (final) teeth alignment obtained from the described inference process. The first row of each example is the frontal view of the same case and corresponding labels in the second row.

FIG. 8A illustrates a front view 802 and a top view 808 of an initial treatment position, a front view 804 and a top view 810 of an actual final treatment position, and a front view 806 and a top view 812 of an intended final treatment position.

FIG. 8B illustrates a front view 822 and a top view 828 of an initial treatment position, a front view 824 and a top view 830 of an actual final treatment position, and a front view 826 and a top view 832 of an intended final treatment position.

FIG. 8C illustrates a front view 842 and a top view 848 of an initial treatment position, a front view 844 and a top view 850 of an actual final treatment position, and a front view 846 and a top view 852 of an intended final treatment position.

FIG. 8D illustrates a front view 862 and a top view 868 of an initial treatment position, a front view 864 and a top view 870 of an actual final treatment position, and a front view 866 and a top view 872 of an intended final treatment position.

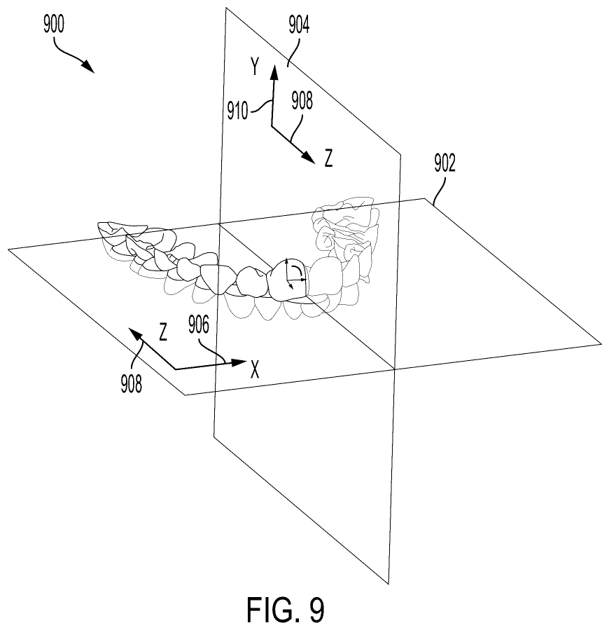

FIG. 9 illustrates an orthodontic arrangement 900 with canonical horizontal and canonical vertical reference planes, in accordance with some embodiments of the technology described herein. Canonical reference planes provide common reference points from which the position and/or orientation of the teeth may be determined such that each of the teeth share a common reference basis. As shown in FIG. 9, canonical horizontal plane 902 is orthogonal to canonical vertical plane 904. Horizontal plane 902 is the plane formed by canonical x-axis 906 and canonical z-axis 908. Vertical plane 904 is the plane formed by canonical y-axis 910 and canonical z-axis 908. In some embodiments, the facial midline is aligned with the canonical y-axis. In some embodiments, the canonical axes are determined from the facial geometry of the patient. In some embodiments, the canonical axes are determined from user input. In some embodiments, the canonical axes are estimated from the initial positions of the teeth.

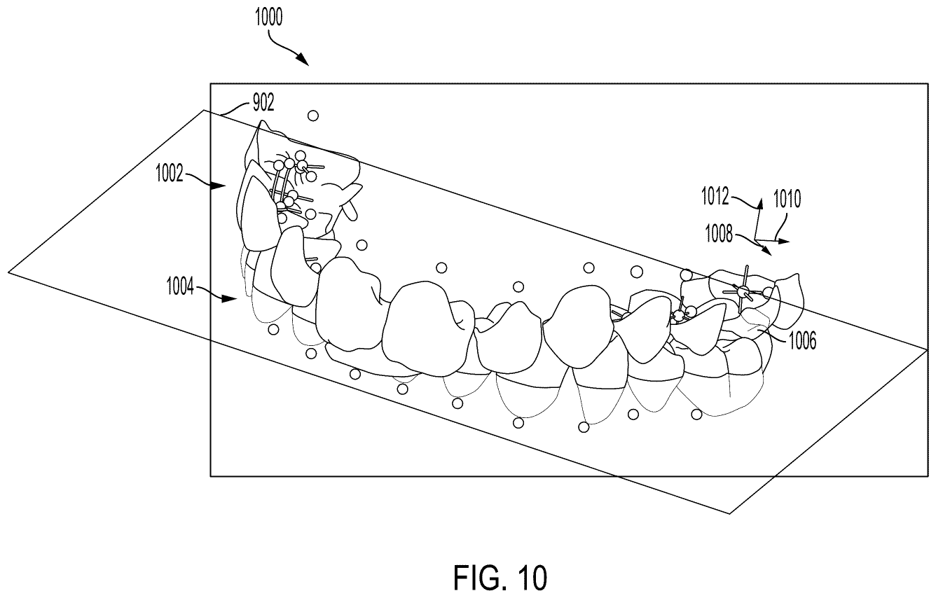

FIG. 10 illustrates a treatment configuration 1000 displayed as part of a GUI for user review and/or adjustment, in accordance with some embodiments of the technology described herein. The GUI display includes horizontal plane 902 such that a user reviewing the treatment configuration 1000 may review the position and orientation of the teeth relative to the horizontal plane 902 as a reference for the alignment. The treatment configuration 1000 includes both a top set of teeth 1002 and a bottom set of teeth 1004.

Any suitable control may be used to provide a user with input controls with which to adjust the position of teeth in the treatment configuration. In the illustrated example of FIG. 10, local axes corresponding to tooth 1006 are included to provide a user with input controls for adjusting the position and/or orientation of tooth 1006. The local axes include local z-axis 1008, local x-axis 1010, and local y-axis 1012. For example, a user may select one of the local axes (e.g., through clicking on it) and may drag the tooth along the axis. Additionally, or alternatively, the user may rotate the tooth around the local axis.

In some embodiments, the local axes are initialized automatically based on feature points of the teeth. In some embodiments, the placement of the local axes relative to the geometry of the tooth may be adjusted by a user.

FIG. 11 illustrates an example of input controls 1100 that may be used in connection with a GUI for adjusting the position and/or orientation of a tooth, in accordance with some embodiments of the technology described herein. Input controls 1100 includes local y-axis 1102, local x-axis 1104, and local z-axis 1106. Each of the local axes includes a ring encircling axis such that a user may select (e.g., by clicking on) the ring to rotate the tooth around the selected axis. The placement of the input controls is based on feature points associated with the tooth 1101. Tooth 1101 is approximated by a geometric block in FIG. 11 including several feature points to illustrate the placement of the local axes. Tooth 1101 includes incisal point 1108, center point 1110, mesial/distal point 1112, facial/lingual point 1114, and center of resistance 1116. As shown in FIG. 11, local y-axis is an axis that includes the incisal 1008 and center 1110 points. Local x-axis is an axis that includes center 1110 and mesial/distal 1112 points. Local z-axis is an axis that includes center 1110 and facial/lingual 1114 points.

The feature points may be determined in any suitable way. In some embodiments, a feature point identification module is used to identify the location of feature points for respective teeth. A process for automatically identifying the location of feature points is described in U.S. patent Publication application Ser. No. “18,535,995”, filed on Dec. 11, 2023, and entitled “TECHNIQUES FOR IDENTIFYING ANATOMICAL DENTAL FEATURE POINTS AND RELATED SYSTEMS AND METHODS,” which is incorporated by reference herein in its entirety.

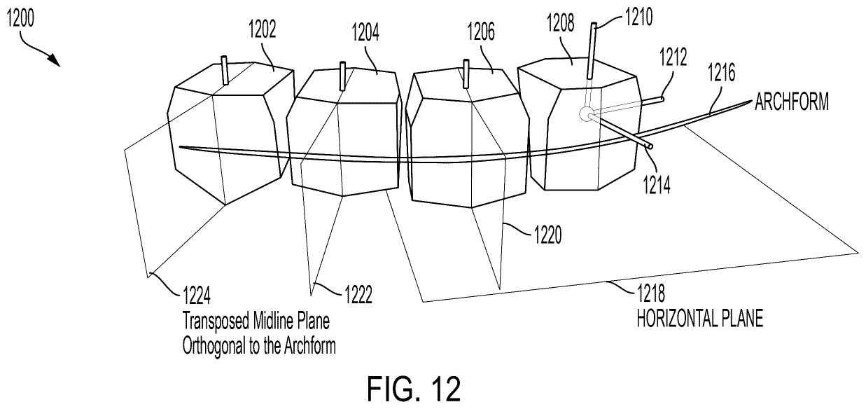

FIG. 12 illustrates an example of an initial treatment configuration 1200 based on a neutral prescription, in accordance with some embodiments of the technology described herein. Initial treatment configuration 1200 includes teeth 1202, 1204, 1206, and 1208. Although four teeth are illustrated, treatment configurations are not limited to four teeth and could include any subset of a patient's teeth and/or prosthetics, including all of a patient's teeth. Each of the teeth includes its own local axes. For example, tooth 1208 includes local y-axis 1210, local x-axis 1212, and local z-axis 1214. The local axes placement is determined based on the geometry of the teeth (e.g., based on the feature points of the teeth), as described above in connection with FIG. 11.

As shown in FIG. 12, the four teeth are positioned relative to an archform 1216. In the neutral position, the teeth are oriented such that their local x-axis and local z-axis are parallel to the canonical horizontal plane 1218. The canonical horizontal plane is determined from the canonical axes, as described above in connection with FIG. 9.

Additionally, in the neutral prescription the teeth are oriented such that a vertical midplane for each tooth, which is oriented parallel to the local z-axis and the local y-axis, is orthogonal to the archform. As shown in FIG. 12—tooth 1202 includes vertical midplane 1224, tooth 1204 includes vertical midplane 1222, and tooth 1206 includes vertical midplane 1220. For the neutral prescription, the spacing between teeth may be taken to be zero, such that there is no gap between adjacent edges of teeth. The neutral prescription may be taken as a zero point for the six canonical coordinates for each tooth. Accordingly, each of the six canonical coordinates may be measure relative to the position of respective teeth in the neutral prescription. In the neutral prescription, the local y-axis is aligned parallel to the canonical y-axis.

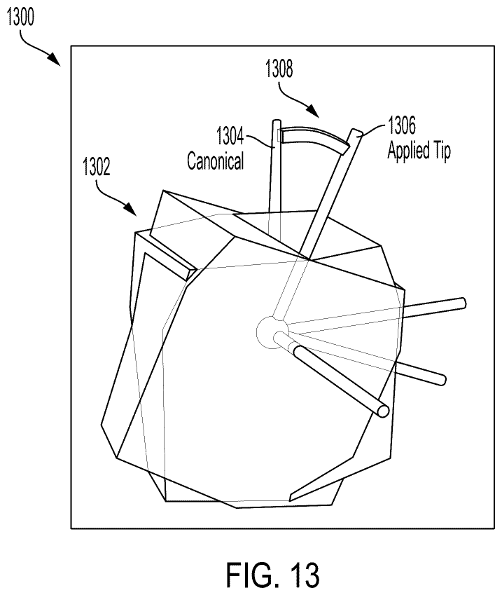

FIG. 13 illustrates an example of a tooth orientation 1300 with a tip applied to the tooth, in accordance with some embodiments of the technology described herein. Tooth orientation 1300 for tooth 1302 includes an applied tip 1308. The applied tip 1308 is measured between the local y-axis 1306 and the canonical y-axis 1304.

FIG. 14 illustrates an example of a GUI 1400 for midline and horizontal plane detection, in accordance with some embodiments of the technology described herein. GUI 1400 includes midline 1402 and horizontal plane 1404. Midline 1402 and horizontal plane 1404 may be automatically determined based on the camera set up. For example, during acquisition of the images of the patient's teeth, the user may be prompted to take a photo with the patient's teeth centered. The user may also be prompted to take a photo with the patient's head in a neutral position such that the patient's upper and lower jaw are aligned with the horizontal plane. As such, the midline 1402 and horizontal plane may be automatically determined based on one or more acquired images. For example, when the photo is taken with the patient's teeth centered, a vertical line passing through the center of the image may be used for the position of the midline. Similarly, a horizontal line passing through the center of the image may be used for the position of the horizontal plane.

GUI 1400 includes a side menu for selecting different aspects of the orthodontic treatment plan to configure. As shown in FIG. 14, the GUI includes a tab 1406 for face setup. Under face setup, the planes which are relevant for the alignment of the teeth relative to the rest of the patient's face are determined and/or adjusted. The planes are relevant for the aesthetic of the treatment. For example, proper aesthetic includes that the arches for the top and lower teeth both be centered on the midline of the face.

In some embodiments, the midplane and/or horizontal plane may be determined based on an arch that describes the tooth positions. For example, an arch may be generated based on the positions of the upper and/or lower teeth such that the arch represents the curvature of the teeth. Based on the determined arch(s), the midplane may be determined by the apex of the arch. The horizontal plane may be determined to be parallel to the plane formed by the arch.

Users may adjust the orientation of the planes. In some embodiments, the user may select the midplane or the horizontal plane through the GUI and may change the position of the planes. In some embodiments, the user may select a photo, from the acquired photos of the patient's teeth, to use as a reference photo. From the reference photo, the midline and/or horizontal planes may be automatically determined, as described herein.

The GUI 1400 includes input features for controlling whether the midline and/or horizontal plane are displayed. In some embodiments, the input features may be check boxes, dropdown menus, buttons, or any other suitable input feature. In the illustrated example of FIG. 14, the GUI includes check box 1408 for controlling whether the midline is displayed and check box 1410 for controlling whether the horizontal plane is displayed.

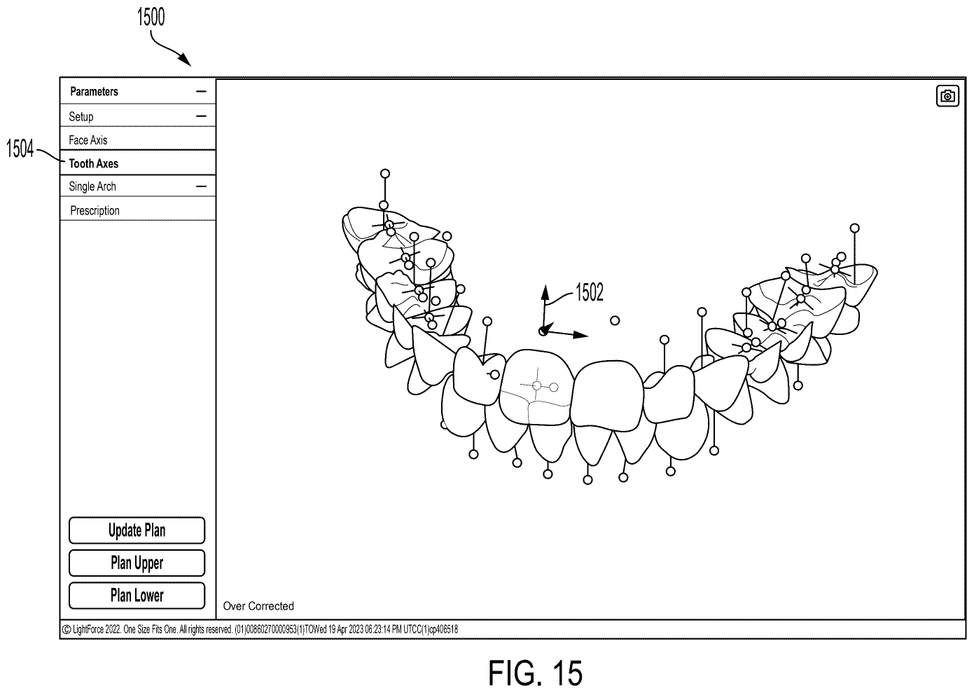

FIG. 15 illustrates an example of a GUI 1500 for tooth axes configuration, in accordance with some embodiments of the technology described herein. GUI 1500 includes tab 1504 in the side panel for selecting tooth axes for review and a modification interface. The modification interface shows the teeth and their corresponding axes. The tooth axes are configured to be aligned with feature points of the teeth, as described herein in connection with FIG. 11 above. Through GUI 1500, a user may adjust the positioning of the local axes of the teeth. A user may select one or more of the local axes and change its positioning to modify the configuration of the local axes for the tooth. Each local axis should be perpendicular to the other axes; therefore, user adjustments to one local axis may impact the other local axes.

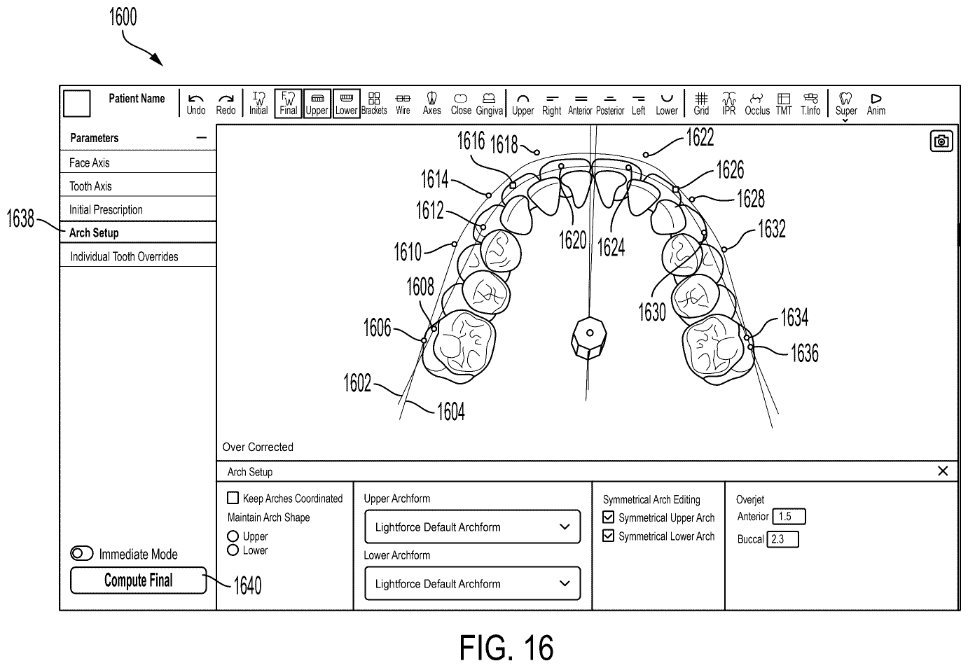

FIG. 16 illustrates an example of a GUI 1600 for arch setup, in accordance with some embodiments of the technology described herein. GUI 1600 includes tab 1638 in the side panel for selecting the arch setup interface. GUI 1600 includes bottom archline 1602 and top archline 1604. The two archlines and corresponding sets of teeth are shown overlaid with each other in the GUI such that a user can compare the positioning and orientation of the teeth in the top and bottom jaws.

Bottom archline 1602 includes multiple control points which represent the geometry of the curve of the archline. The control points may be selected by a user and their position modified to change the shape of the arch such that the arch passes through the modified position of the control point. After the user has adjusted the control points, the user may select the compute final button 1640 to initiate the process for computing an updated tooth configuration based on the archlines.

As shown in FIG. 16, bottom archline includes control points 1608, 1612, 1616, 1620, 1624, 1626, 1630, and 1634. Similarly, top archline 1604 includes control points 1606, 1610, 1614, 1618, 1622, 1628, 1632, and 1636. Although in the illustrated embodiments each archline is shown as having eight control points, the archlines may have other numbers of control points and/or the top and bottom archline may have different numbers of control points, respectively.

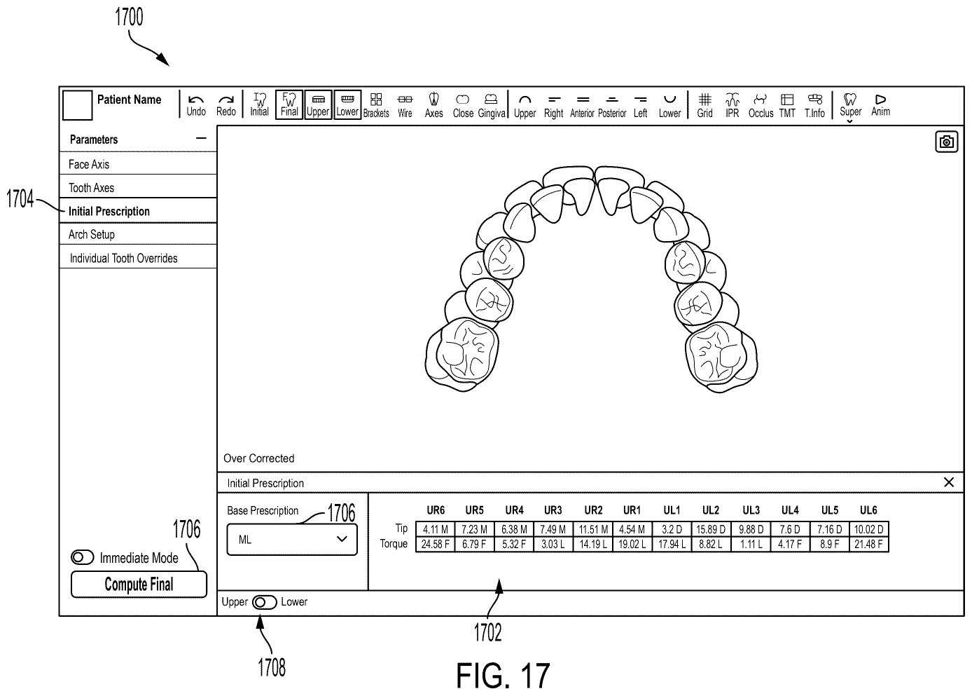

FIG. 17 illustrates an example of a GUI 1700 for showing an initial prescription, in accordance with some embodiments of the technology described herein. GUI 1700 includes tab 1704 in the side panel for selecting the arch setup interface. Table 1702 displays initial prescription values associated with the selected initial prescription. The table includes a column for each tooth in the upper jaw and corresponding rows for the prescription coordinates. A corresponding table for the lower jaw can be displayed with toggle control 1708. The initial prescription selection may be changed with drop down menu 1708.

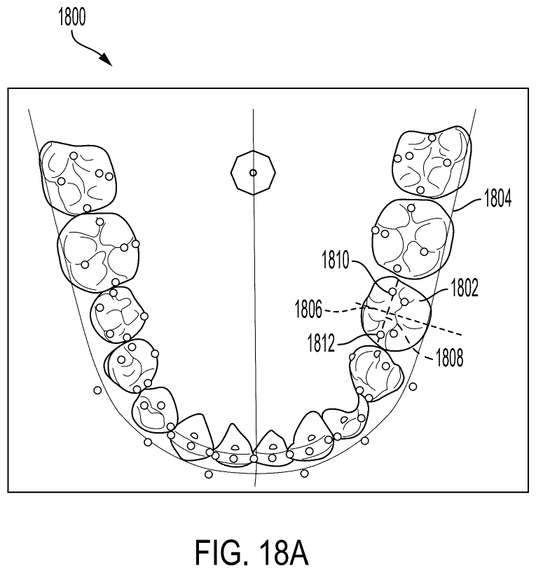

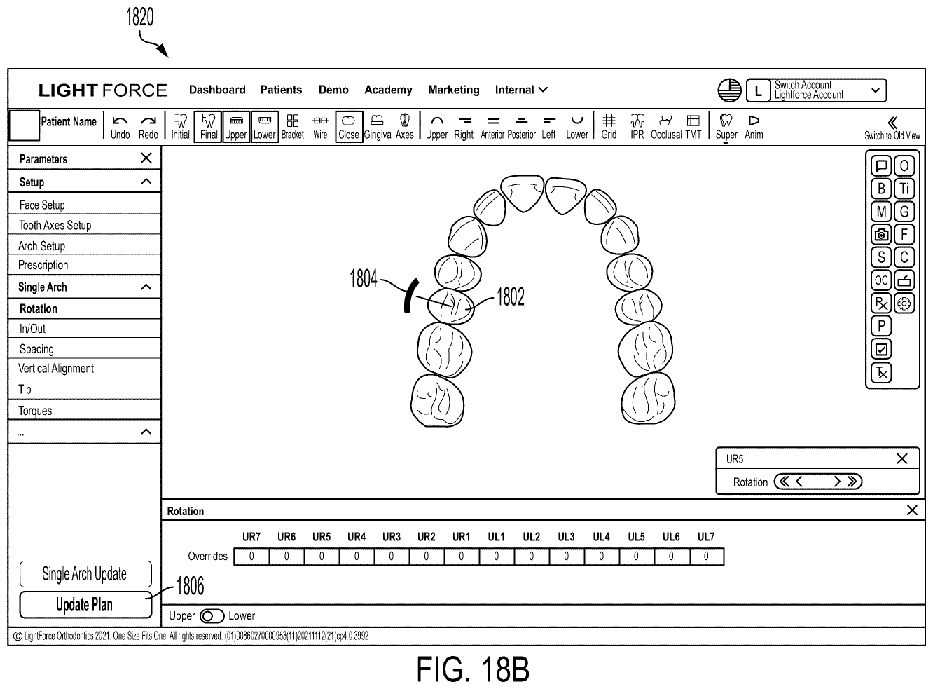

FIG. 18A illustrates an example of a GUI 1800 for showing the initial rotation for a patient's teeth, in accordance with some embodiments of the technology described herein. GUI 1800 includes control points for adjusting archform 1804. Upon adjustments to the archform, the teeth are automatically aligned to the archform based on the feature points of the teeth. Tooth 1802 includes proximal feature points 1810 and 1812 through which a center line 1808 passes. A second line 1806 passes through the center of center line 1808 perpendicular to the center line. Tooth 1802 is moved aligned such that center line 1808 is tangent to the archform 1804 and moved along second line 1806 to the desired offset from the archform 1804. In the illustrated example, the desired offset is zero for tooth 1802.

FIG. 18B illustrates an example of a GUI 1820 for rotation of a tooth, in accordance with some embodiments of the technology described herein. As shown in FIG. 18B, tooth 1802 is selected and arch 1804 may be selected and dragged to rotate tooth 1802. After the tooth has been rotated by a user, the user may select update plan button 1806 to update the tooth rotation in the prescription and update the display to reflect the new positions of each of the teeth to accommodate the updated rotation of tooth 1804.

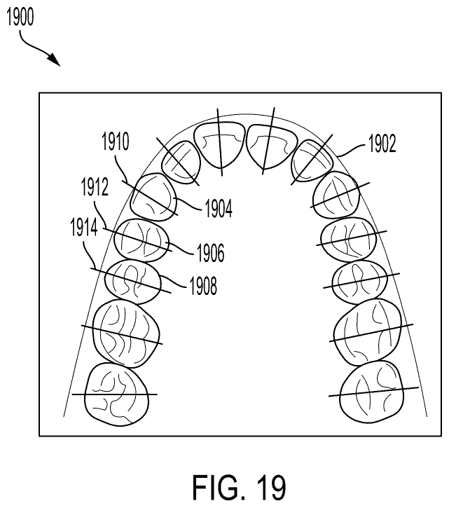

FIG. 19 illustrates an example of a GUI 1900 for adjusting the in/out distance of a tooth, in accordance with some embodiments of the technology described herein. GUI 1900 includes archform 1902, and teeth 1904, 1906, and 1908. The teeth include lines 1910, 1912, and 1914 for adjusting teeth 1904, 1906, and 1908 respectively. A user may select one of the lines 1910, 1912, or 1914 to click and drag to move the respective tooth along the line to adjust the in/out distance of the tooth from the archform 1902.

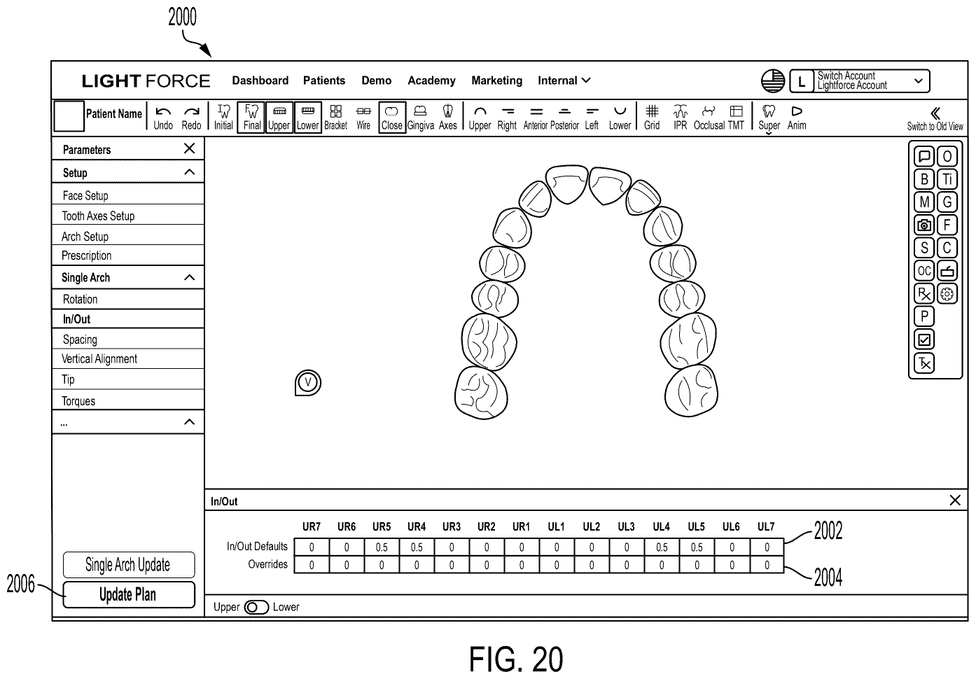

FIG. 20 illustrates a second example of a GUI 2000 for adjusting the in/out distance of a patient's teeth, in accordance with some embodiments of the technology described herein. GUI 2000 includes a prescription table 2002 with a column corresponding to each tooth in the upper jaw of a patient. GUI 2000 includes override table 2004 for a user to input changes to the in/out distance of the prescription. After overrides are entered into the table, the user may select the update plan button 2006 to update the prescription and tooth positions.

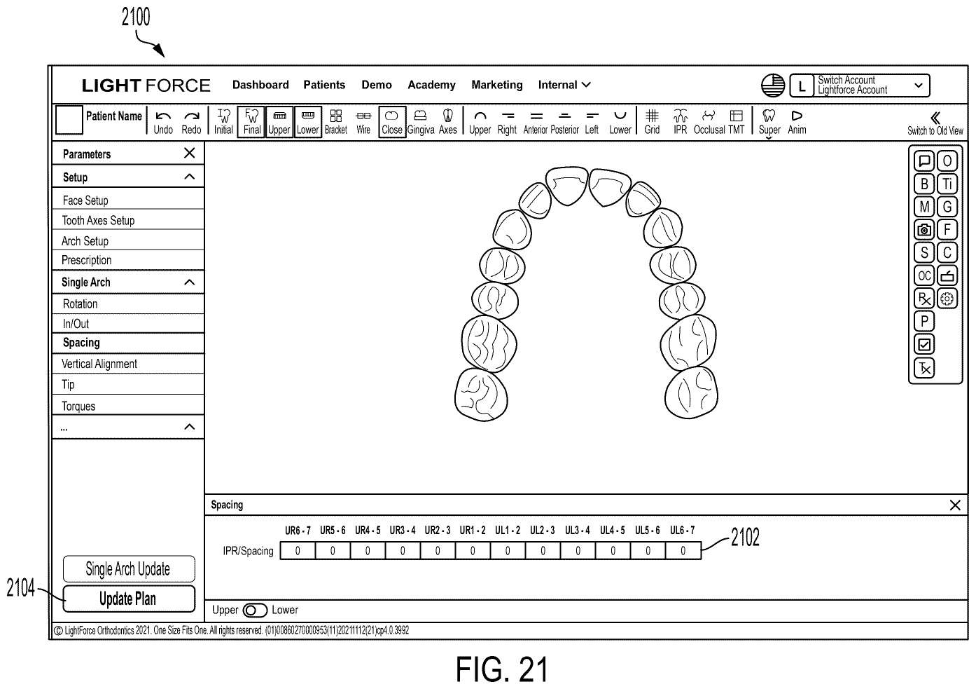

FIG. 21 illustrates a second example of a GUI 2100 for adjusting the IPR/spacing distance of a patient's teeth, in accordance with some embodiments of the technology described herein. GUI 2100 includes a prescription table 2102 with a column corresponding to each tooth in the upper jaw of a patient. GUI 2100 includes override table 2004 for a user to input changes to the IPR distance/spacing prescription. After overrides are entered into the table, the user may select the update plan button 2006 to update the prescription and tooth positions.

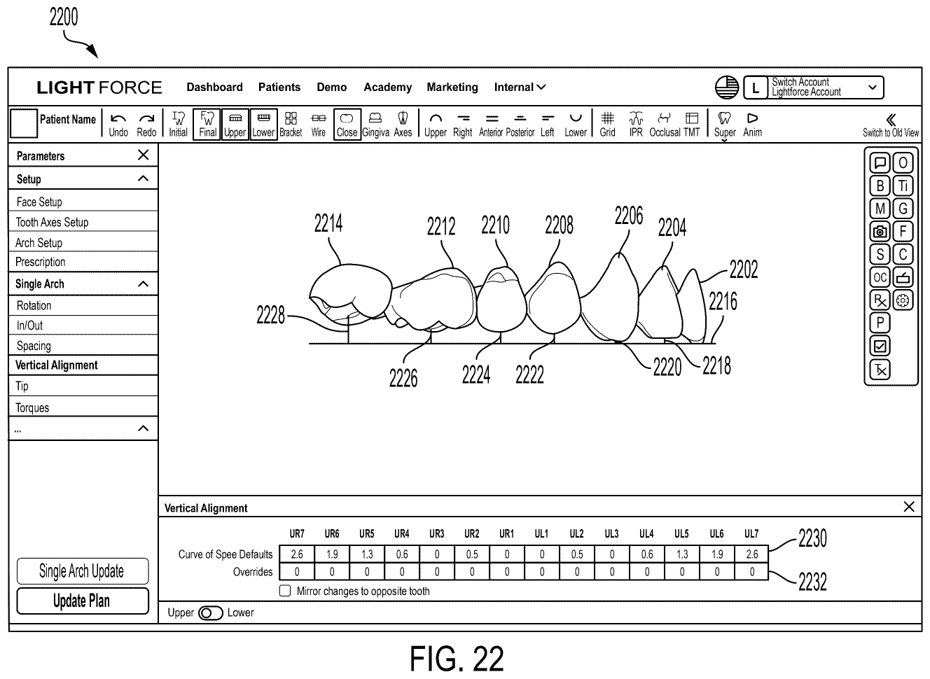

FIG. 22 illustrates an example of a GUI 2200 for adjusting the height of the teeth relative to the archform, in accordance with some embodiments of the technology described herein. GUI 2200 includes a prescription table 2230 with a column corresponding to each tooth in the upper jaw of a patient. GUI 2200 includes override table 2232 for a user to input changes to the height of the teeth relative to the archform in the prescription. The tooth heights may be automatically determined for the prescription to increase heights from the front teeth to the back teeth. For example, from front to back, GUI 2200 shows teeth 2202, 2204, 2206, 2208, 2210, 2212, and 2214.

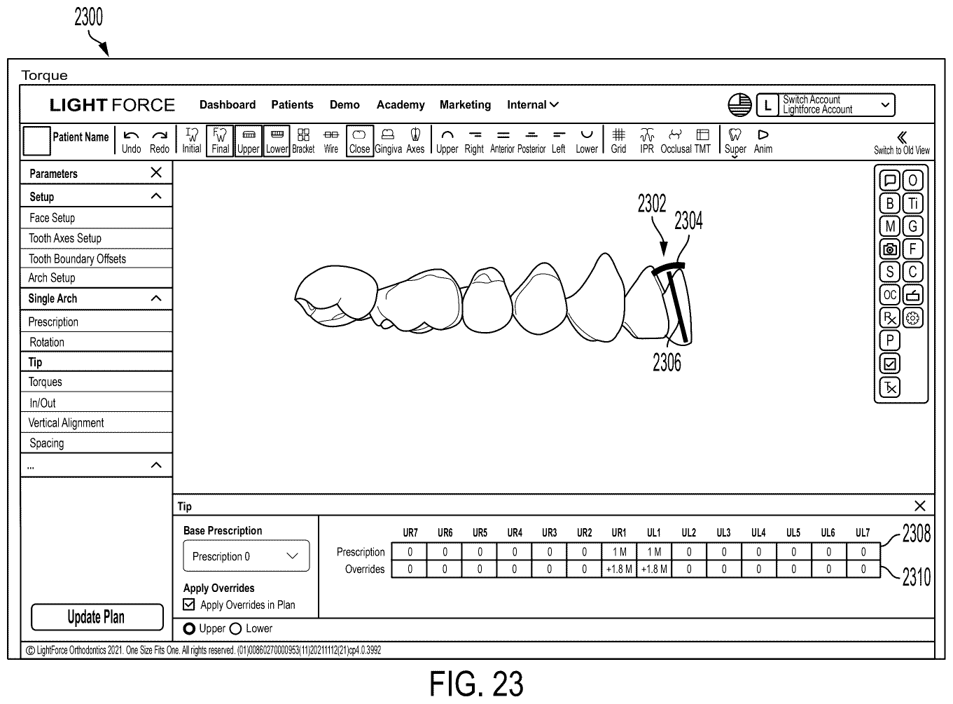

FIG. 23 illustrates an example of a GUI 2300 for adjusting the tilt of the teeth, in accordance with some embodiments of the technology described herein. GUI 2300 includes a prescription table 2208 with a column corresponding to each tooth in the upper jaw of a patient. GUI 2300 includes override table 2310 for a user to input changes to the tilt of the teeth relative to the canonical axes. As shown in FIG. 23, the tilt may be adjusted such that the surface of the tooth is tilted from a first position 2304 to 2306, where the difference between the first position and the second position is angle 2302.

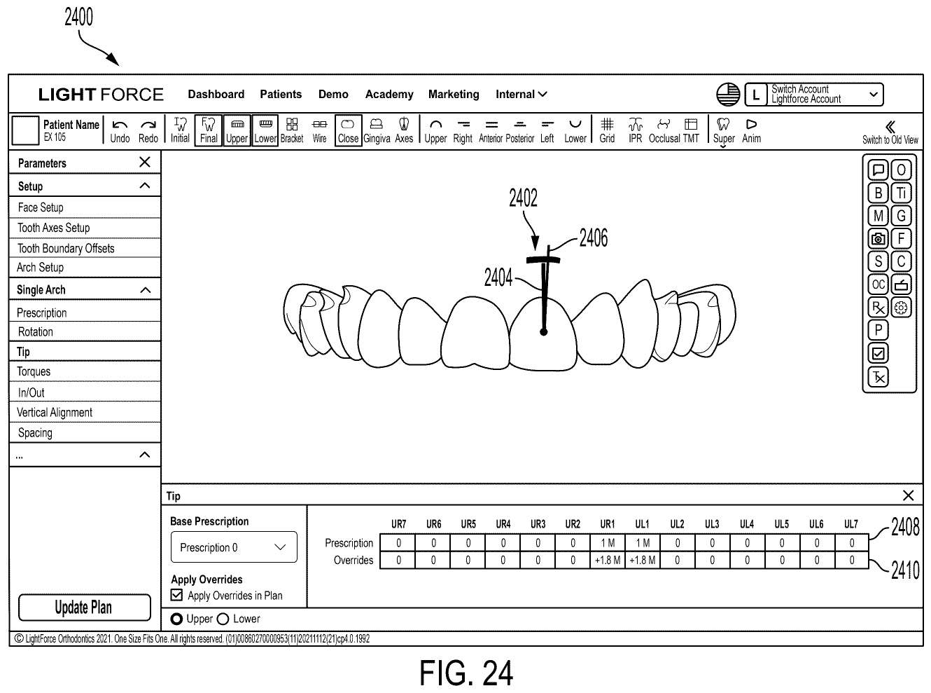

FIG. 24 illustrates an example of a GUI 2400 for adjusting the torque of the teeth, in accordance with some embodiments of the technology described herein. GUI 2400 includes a prescription table 2408 with a column corresponding to each tooth in the upper jaw of the patient. GUI 2400 includes override table 2410 for a user to input changes to the torque of the teeth. As shown in FIG. 24, the torque of the teeth may be adjusted from a first angle 2404 to a second angle 2406 where the difference between those angles is 2402.

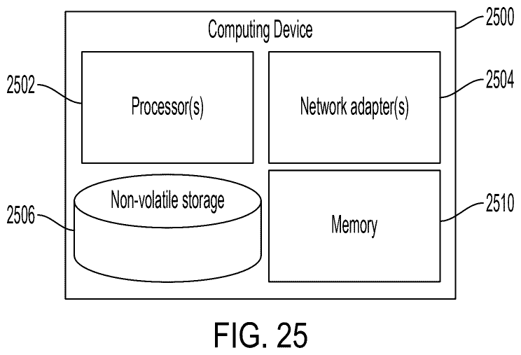

FIG. 25 illustrates an example implementation of a computer system 2500 that may be used in connection with any of the embodiments of the disclosure provided herein. For example, the processes described herein may be performed using computing system 2500. The computing system 2500 may include one or more computer hardware processors 2502 and one or more non-transitory computer-readable storage media. For example, one or more volatile storage devices 2510 and/or one or more non-volatile storage devices 2506 (e.g., a hard disk, a flash memory, etc.) may be included with computing system 2500. The hardware processor 2502 may control writing data to and reading data from the volatile storage device 2510 and the non-volatile storage device 2506, which may serve as non-transitory computer-readable storage media storing processor-executable instructions for execution by the hardware processor 2502.

Computing system 2500 may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, tablet, or any other suitable portable or fixed electronic device, as aspects of the technology described herein are not limited in this respect.

Also, a computer may have one or more input and output devices. These devices may be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks, fiber optic networks, or any suitable combination thereof. As shown in FIG. 25, computing system 2500 includes network adapter(s) 2504 to facilitate network connectivity.

Having thus described several aspects of at least one embodiment of the technology described herein, it is to be appreciated that various alterations, modifications, and improvements will readily occur to those skilled in the art.

Such alterations, modifications, and improvements are intended to be part of this disclosure and are intended to be within the spirit and scope of disclosure. Further, though advantages of the technology described herein are indicated, not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein may be implemented in any of numerous ways. For example, the embodiments may be implemented using hardware, software, or a combination thereof. When implemented in software, the software code may be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media may be transportable, such that the program or programs stored thereon may be loaded onto one or more different computers or other processors to implement various aspects of the technology as described above. As used herein, the term “computer-readable storage medium” encompasses only a non-transitory computer readable medium that may be considered to be a manufacture (i.e., article of manufacture) or a machine.

The terms “program” or “software” are used herein in a generic sense to refer to any type of computer code or set of processor-executable instructions that may be employed to program a computer or other processor to implement various aspects of the technology as described above. Additionally, one or more computer programs that when executed perform methods of the technology described herein need not reside on a single computer or processor, but may be distributed in a modular fashion amongst a number of different computers or processors to implement various aspects of the technology described herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving,” and variations thereof, is meant to encompass the items listed thereafter and additional items.

Unless otherwise specified, the terms “approximately,” “substantially,” and “about” may be used to mean within +10% of a target value in some embodiments. The terms “approximately,” “substantially” and “about” may include the target value.

Various aspects are described in this disclosure, which include, but are not limited to the following aspects:
1. A method for use by an orthodontic treatment platform for generating an orthodontic treatment plan, the method comprising: executing the orthodontic treatment platform using at least one computer hardware processor to perform: obtaining an initial treatment configuration comprising anatomical representations of a patient's teeth; obtaining an initial prescription, the initial prescription comprising spatial coordinates for modeling positions of a patient's teeth, the spatial coordinates being relative to a canonical coordinate system; determining an alignment of the patient's teeth based on the initial treatment configuration and the initial prescription; outputting the alignment of the patient's teeth through a graphical user interface; receiving an updated spatial coordinate for one or more of the patient's teeth through the graphical user interface; and generating an updated prescription by updating one or more spatial coordinates of the initial prescription based on the updated spatial coordinate.
2. The method of aspect 1, wherein the spatial coordinates of the initial prescription comprise position coordinates and orientation coordinates, each of the position coordinates and the orientation coordinates are measured relative to a common reference coordinate for each of the patient's teeth.
3. The method of any of aspects 1-2, wherein the graphical user interface displays one or more axis of a tooth for adjusting the spatial coordinates of the tooth, the one or more axis of the tooth being based on a shape of the tooth.
4. The method of any of aspects 1-3, wherein updating the one or more spatial coordinates comprises generating a separate prescription associated with a user of the graphical user interface.
5. The method of any of aspects 1-4, wherein generating the updated orthodontic treatment plan based on the updated one or more spatial coordinates further comprises determining positions for each of the patient's teeth to accommodate the updated target tooth position while maintaining the spatial coordinates specified by the prescription.
6. An orthodontic treatment platform comprising at least one computer hardware processor and at least one non-transitory computer-readable storage medium storing processor executable instructions that when executed by the at least one computer hardware processor perform a method of any one of aspects 1-5.
7. At least one non-transitory computer-readable storage medium storing processor executable instructions that when executed by the at least one computer hardware processor perform a method of any one of aspects 1-5.
8. A method for use by an orthodontic treatment platform for updating an orthodontic treatment plan for a patient, the method comprising: executing the orthodontic treatment platform using at least one computer hardware processor to perform: receiving an updated target tooth position based on an adjustment to a planned target tooth position, the planned target tooth position being associated with the orthodontic treatment plan; updating one or more spatial coordinates of a prescription based on the received adjustment, the one or more spatial coordinates being relative to a canonical coordinate system; determining an updated orthodontic treatment plan based on the updated one or more spatial coordinates; and displaying the updated orthodontic treatment plan through a graphical user interface.
9. The method of aspect 8, wherein the spatial coordinates of the prescription comprise position coordinates and orientation coordinates, each of the position coordinates and the orientation coordinates are measured relative to a common reference coordinate for each of the patient's teeth.
10. The method of any of aspects 8-9, wherein receiving the updated target tooth position comprises receiving the adjustment from the graphical user interface.
11. The method of aspect 10, wherein the graphical user interface displays one or more axis of a tooth for adjusting the spatial coordinates of the tooth, the axis of the tooth being based on a shape of the tooth.
12. The method of any of aspects 8-11, wherein updating the one or more spatial coordinates comprises generating a separate prescription associated with a user of the graphical user interface.
13. The method of any of aspects 8-12, wherein determining the updated orthodontic treatment plan based on the updated one or more spatial coordinates comprises determining positions for each of the patient's teeth to accommodate the updated target tooth position while maintaining the one or more spatial coordinates specified by the prescription.
14. An orthodontic treatment platform comprising at least one computer hardware processor and at least one non-transitory computer-readable storage medium storing processor executable instructions that when executed by the at least one computer hardware processor perform a method of any one of aspects 8-13.
15. At least one non-transitory computer-readable storage medium storing processor executable instructions that when executed by the at least one computer hardware processor perform a method of any one of aspects 8-13.
16. A method for use by an orthodontic treatment platform for determining an alignment of patient teeth based on an orthodontic prescription using a plurality of alignment operations, the method comprising: executing the orthodontic treatment platform using at least one computer hardware processor to perform: obtaining an initial treatment configuration comprising anatomical representations of a patient's teeth; obtaining an adjustment to a target tooth position associated with an orthodontic treatment plan; obtaining a prescription, the prescription comprising spatial coordinates for modeling the position of a patient's teeth, the spatial coordinates being relative to a canonical coordinate system; and determining a plurality of alignment operations, based on the prescription, to align the patient's teeth to a treatment configuration, the treatment configuration.
17. The method of aspect 16, wherein determining the plurality of alignment operations comprises determining placement of the patient's teeth sequentially from front teeth to back teeth.
18. The method of any of aspects 16-17, wherein the spatial coordinates of the prescription comprise position coordinates and orientation coordinates, each of the position coordinates and the orientation coordinates are measured relative to a common reference coordinate for each of the patient's teeth.
19. The method of aspect 18, wherein determining the plurality of alignment operations comprises aligning the target tooth to one or more position coordinates, the aligning comprising: determining an arch distance between the target tooth position and an archform; determining a spacing between the target tooth position and a neighboring tooth; and determining a vertical offset between the target tooth position and an initial vertical position.
20. The method of aspect 19, wherein determining the plurality of alignment operations comprises aligning a height of each of the patient's teeth in the orthodontic treatment plan such that the vertical offset of the patient's teeth, relative to a horizontal plane, increases from front teeth to back teeth.
21. The method of aspect 19, wherein determining a spacing between the target tooth position and a neighboring tooth comprises determining a distance between a first feature point corresponding to an edge of the target tooth and a second feature point corresponding to the edge of the neighboring tooth.
22. The method of aspect 18, wherein updating the orientation coordinates of the spatial coordinates using a plurality of treatment operations, the plurality of treatment operations comprising: determining a tip rotation about a canonical z-axis by determining an angle between a y axis of the target tooth and a canonical y-axis along a XY plane of the target tooth; determining a torque rotation about a canonical x-axis by determining an angle between a y-axis of the target tooth and the canonical y-axis along a YZ plane of the target tooth; and determining a tooth rotation about a canonical y-axis by determining an angle between the z-axis of the target tooth and the canonical z-axis along a XZ plane of the target tooth.
23. The method of aspect 22, wherein determining the plurality of treatment operations comprises applying transformations relative to the canonical coordinates, the transformations corresponding to the orientation coordinates being applied in a predetermined sequence.
24 The method of aspect 23, wherein the predetermined sequence comprises: applying the tooth rotation about the canonical y-axis of the target tooth; napplying the tip rotation about the canonical z-axis, subsequent to applying the tooth rotation about the canonical y-axis; and applying the torque rotation about the canonical x-axis, subsequent to applying the tip rotation.
25. The method of any of aspects 18-24, wherein the orientation coordinates are updated prior to updating the position coordinates.
26. An orthodontic treatment platform comprising at least one computer hardware processor and at least one non-transitory computer-readable storage medium storing processor executable instructions that when executed by the at least one computer hardware processor perform a method of any one of aspects 16-25.
27 At least one non-transitory computer-readable storage medium storing processor executable instructions that when executed by the at least one computer hardware processor perform a method of any one of aspects 16-25.
28. A method for using a trained machine learning (ML) model to determine an orthodontic treatment plan configuration, the method comprising: executing an orthodontic treatment platform using at least one computer hardware processor to perform: obtaining a first treatment configuration and prescription; and processing the first treatment configuration and the prescription using the trained ML model to determine a second treatment configuration, the processing comprising: encoding three-dimensional spatial data as respective tooth data; determining an arrangement for a patient's teeth using the trained ML model to process the encoded three-dimensional spatial data; determining canonical coordinates for the patient's teeth based on the determined arrangement; and determining tooth positions based on the first treatment configuration and the canonical coordinates.
29. The method of aspect 28, wherein the trained ML model comprises between 100 million and 400 million trainable parameters.
30 The method of any of aspects 28-29, wherein the three-dimensional spatial data is point cloud data and encoding the point cloud comprises generating vectors representing a shape of respective teeth from spatial data in the point cloud.
31. The method of aspect 30, further comprising generating feature vectors that are representative of the determined arrangement for the patient's teeth.
32. The method of aspect 31, wherein the encoded three-dimensional tooth data is combined with the feature vectors.
33. The method of any of aspects 28-32, wherein determining the arrangement for the teeth comprises using a transformer model configured to process the encoded three-dimensional data and a relationship of a first tooth to neighboring teeth.
34 The method of aspect 33, wherein the relationship of the first tooth to the neighboring teeth is a spatial relationship between the first tooth and a second tooth next to the first tooth in a same jaw as the first tooth.
35. The method of aspect 33, wherein the relationship of the first tooth to the neighboring teeth is a spatial relationship between the first tooth and each other tooth in a same jaw as the first tooth.
36. An orthodontic treatment platform comprising at least one computer hardware processor and at least one non-transitory computer-readable storage medium storing processor executable instructions that when executed by the at least one computer hardware processor perform a method of any one of aspects 28-35.
37. At least one non-transitory computer-readable storage medium storing processor executable instructions that when executed by the at least one computer hardware processor perform a method of any one of aspects 28-35.

1. A method for use by an orthodontic treatment platform for determining an alignment of patient teeth based on an orthodontic prescription using a plurality of alignment operations, the method comprising:
executing the orthodontic treatment platform using at least one computer hardware processor to perform:
obtaining an initial treatment configuration comprising anatomical representations of a patient's teeth;
obtaining an adjustment to a target tooth position associated with an orthodontic treatment plan;
obtaining a prescription, the prescription comprising spatial coordinates for modeling the position of a patient's teeth, the spatial coordinates being relative to a canonical coordinate system; and
determining a plurality of alignment operations, based on the prescription, to align the patient's teeth to a treatment configuration, the treatment configuration.

2. The method of claim 1, wherein determining the plurality of alignment operations comprises determining placement of the patient's teeth sequentially from front teeth to back teeth.

3. The method of claim 1, wherein the spatial coordinates of the prescription comprise position coordinates and orientation coordinates, each of the position coordinates and the orientation coordinates are measured relative to a common reference coordinate for each of the patient's teeth.

4. The method of claim 3, wherein determining the plurality of alignment operations comprises aligning the target tooth to one or more position coordinates, the aligning comprising:
determining an arch distance between the target tooth position and an archform;
determining a spacing between the target tooth position and a neighboring tooth; and
determining a vertical offset between the target tooth position and an initial vertical position.

5. The method of claim 4, wherein determining the plurality of alignment operations comprises aligning a height of each of the patient's teeth in the orthodontic treatment plan such that the vertical offset of the patient's teeth, relative to a horizontal plane, increases from front teeth to back teeth.

6. The method of claim 4, wherein determining a spacing between the target tooth position and a neighboring tooth comprises determining a distance between a first feature point corresponding to an edge of the target tooth and a second feature point corresponding to the edge of the neighboring tooth.

7. The method of claim 3, wherein updating the orientation coordinates of the spatial coordinates using a plurality of treatment operations, the plurality of treatment operations comprising:
determining a tip rotation about a canonical z-axis by determining an angle between a y axis of the target tooth and a canonical y-axis along a XY plane of the target tooth;
determining a torque rotation about a canonical x-axis by determining an angle between a y-axis of the target tooth and the canonical y-axis along a YZ plane of the target tooth; and
determining a tooth rotation about a canonical y-axis by determining an angle between the z-axis of the target tooth and the canonical z-axis along a XZ plane of the target tooth.

8. The method of claim 7, wherein determining the plurality of treatment operations comprises applying transformations relative to the canonical coordinates, the transformations corresponding to the orientation coordinates being applied in a predetermined sequence.

9. The method of claim 8, wherein the predetermined sequence comprises:
applying the tooth rotation about the canonical y-axis of the target tooth;
applying the tip rotation about the canonical z-axis, subsequent to applying the tooth rotation about the canonical y-axis; and
applying the torque rotation about the canonical x-axis, subsequent to applying the tip rotation.

10. The method of claim 3, wherein the orientation coordinates are updated prior to updating the position coordinates.

11. An orthodontic treatment platform comprising at least one computer hardware processor and at least one non-transitory computer-readable storage medium storing processor executable instructions that when executed by the at least one computer hardware processor perform a method, the method comprising:
obtaining an initial treatment configuration comprising anatomical representations of a patient's teeth;
obtaining an adjustment to a target tooth position associated with an orthodontic treatment plan;
obtaining a prescription, the prescription comprising spatial coordinates for modeling the position of a patient's teeth, the spatial coordinates being relative to a canonical coordinate system; and
determining a plurality of alignment operations, based on the prescription, to align the patient's teeth to a treatment configuration, the treatment configuration.

12. The orthodontic treatment platform of claim 11, wherein determining the plurality of alignment operations by the at least one computer hardware processor comprises determining placement of the patient's teeth sequentially from front teeth to back teeth.

13. The orthodontic treatment platform of claim 11, wherein the spatial coordinates of the prescription comprise position coordinates and orientation coordinates, each of the position coordinates and the orientation coordinates are measured relative to a common reference coordinate for each of the patient's teeth.

14. The orthodontic treatment platform of claim 13, wherein determining the plurality of alignment operations by the at least one computer hardware processor comprises aligning the target tooth to one or more position coordinates, the aligning comprising:
determining an arch distance between the target tooth position and an archform;
determining a spacing between the target tooth position and a neighboring tooth; and
determining a vertical offset between the target tooth position and an initial vertical position.

15. The orthodontic treatment platform of claim 14, wherein determining the plurality of alignment operations by the at least one computer hardware processor comprises aligning a height of each of the patient's teeth in the orthodontic treatment plan such that the vertical offset of the patient's teeth, relative to a horizontal plane, increases from front teeth to back teeth.

16. The orthodontic treatment platform of claim 14, wherein determining a spacing between the target tooth position and a neighboring tooth by the at least one computer hardware processor comprises determining a distance between a first feature point corresponding to an edge of the target tooth and a second feature point corresponding to the edge of the neighboring tooth.

17. The orthodontic treatment platform of claim 13, wherein updating the orientation coordinates of the spatial coordinates using a plurality of treatment operations, by the at least one computer hardware processor comprises:
determining a tip rotation about a canonical z-axis by determining an angle between a y axis of the target tooth and a canonical y-axis along a XY plane of the target tooth;
determining a torque rotation about a canonical x-axis by determining an angle between a y-axis of the target tooth and the canonical y-axis along a YZ plane of the target tooth; and
determining a tooth rotation about a canonical y-axis by determining an angle between the z-axis of the target tooth and the canonical z-axis along a XZ plane of the target tooth.

18. The orthodontic treatment platform of claim 17, wherein determining the plurality of treatment operations by the at least one computer hardware processor comprises applying transformations relative to the canonical coordinates, the transformations corresponding to the orientation coordinates being applied in a predetermined sequence.

19. The orthodontic treatment platform of claim 18, wherein the predetermined sequence comprises:
applying the tooth rotation about the canonical y-axis of the target tooth;
applying the tip rotation about the canonical z-axis, subsequent to applying the tooth rotation about the canonical y-axis; and
applying the torque rotation about the canonical x-axis, subsequent to applying the tip rotation.

20. At least one non-transitory computer-readable storage medium storing processor executable instructions that when executed by the at least one computer hardware processor perform a method, the method comprising:
obtaining an initial treatment configuration comprising anatomical representations of a patient's teeth;
obtaining an adjustment to a target tooth position associated with an orthodontic treatment plan;
obtaining a prescription, the prescription comprising spatial coordinates for modeling the position of a patient's teeth, the spatial coordinates being relative to a canonical coordinate system; and
determining a plurality of alignment operations, based on the prescription, to align the patient's teeth to a treatment configuration, the treatment configuration.
